SEQUENCE LISTING (10 sequences)

SEQ ID NO: 1 - DNA, 615 bp, Homo sapiens
  source 1..615: mol_type = other DNA
atggctggacctgccacccagagccccatgaagctgatggccctgcagctgctgctgtggcacagtgcactctggacagtgcaggaagccacccccctgggccctgccagctccctgccccagagcttcctgctcaagtgcttagagcaagtgaggaagatccagggggatggggcagctctccaggagaagctgtgtgccacctacaagctgtgccaccctgaggagctggtgctgctgggacactctctgggcatcccctgggctcccctgagcagctgccccagccaggccctgcagctggcaggctgcttgagccaactccatagtggccttttcctctaccaggggctcctgcaggccctggaagggatctcccctgagttgggtcccaccttggacacactgcagctggatgttgctgactttgccaccaccatctggcagcagatggaagaactgggaatggcccctgccctgcagcccacccagggtgccatgcctgcctttgcctctgctttccagagaagggcaggaggggtcctggttgcctcccatctgcagagcttcctggaggtgtcctacagagttctaagacaccttgcccagccctga

SEQ ID NO: 2 - protein, 207 aa, synthetic construct
  source 1..207: note = synthetic
MAGPATQSPMKLMALQLLLWHSALWTVQEATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 3-4 - skipped (2 entries)

SEQ ID NO: 5 - DNA, 4308 bp, synthetic construct
  source 1..4308: mol_type = other DNA; note = synthetic
cactatgtggacatgaattcaattggctagcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttggtaagtcactgactgtctatgcctgggaaagggtgggcaggagatggggcagtgcaggaaaagtggcactatgaaccctgcagccctagacaattgtactaaccttcttctctttcctctcctgacaggttggtaaccaagccaccatgggctggtccctgatcctgctgttcctggtggctgtggccaccagagtgctgagccaggtgcagctgcagcagcctggggctgagcttgtgaaacctggggcctctgtgaagatgagctgcaaggcctctggctacaccttcaccagctacaacatgcactgggtcaagcagacccctggcagaggcctggaatggattggagccatctaccctggcaatggggacacctcctacaaccagaagttcaagggcaaggccaccctgacagctgacaagagcagcagcacagcctacatgcagctgtccagcctgacctctgaggactctgctgtgtactactgtgccaggtccacctactatgggggagactggtacttcaatgtgtggggagctggcaccacagtgacagtgtctgctgccagcaccaagggcccctctgtgtttcctctggcccccagcagcaagagcacctctgggggaacagctgccctgggctgccttgtgaaggactacttccctgagcctgtgactgtgtcctggaactctggggccctgacatctggggtgcacaccttccctgcagtgctgcagtccagtggcctgtactccctgtcctctgttgtgacagtgcccagctccagcctgggcacccagacctacatctgcaatgtgaaccacaagcccagcaacaccaaggtggacaagaaggctgagcccaagagctgtgacaagacccacacctgtcccccctgtcctgcccctgaactgctgggaggaccttctgtgttcctgttcccacccaagcccaaggataccctgatgatcagcagaacccctgaagtgacctgtgtggtggtggatgtgtcccatgaggacccagaagtgaagttcaattggtatgtggatggggtggaagtgcacaatgccaagaccaagcccagagaggaacagtacaacagcacctacagagtggtgtctgtgctgactgtgctgcaccaggactggctgaatggcaaagagtacaagtgcaaggtgtccaacaaggccctgccagcccccattgagaaaaccatcagcaaggccaagggccagcctagagaaccccaggtgtacacactgccccctagcagggatgagctgaccaagaaccaggtgtccctgacatgccttgtgaaaggcttctacccctctgacattgctgtggaatgggagagcaatggacagcctgagaacaactacaagaccaccccccctgtgctggactctgatggctcattcttcctgtacagcaagctgacagtggacaagtccagatggcagcagggcaatgtgttcagctgctctgtgatgcatgaggccctgcacaaccactacacccagaaaagcctgtccctgtcccctggcaagagagcaaagagggcaccagtgaaacagactttgaattttgaccttctcaagttggcaggagatgtggagtccaaccctggacctatggacttccaggtgcagatcatcagctttctgctgatctctgcctctgtgatcatgagcagaggccagattgtgctgagccagagccctgccatcctgtctgcaagccctggggagaaagtgaccatgacctgcagagccagcagctctgtgtcctacatccactggttccagcagaagcctggcagcagccccaagccttggatctatgccaccagcaacctggcatctggggtgccagtcagattctctggctctggatctggcaccagctacagcctgaccatcagcagagtggaagctgaggatgctgccacctactactgccagcagtggaccagcaatccccccacctttggagggggcaccaagctggaaatcaagagaacagtggctgccccctctgtgttcatcttcccaccctctgatgagcagctgaagtctggaacagcctctgttgtgtgcctgctgaacaacttctaccccagagaagccaaggtgcagtggaaggtggacaatgccctgcagtctggcaactcccaggaatctgtgacagagcaggacagcaaggactccacctactccctgagcagcaccctgaccctgagcaaggctgactatgagaagcacaaagtgtatgcctgtgaagtgacccaccagggcctgtccagccctgtgaccaagagcttcaacagaggggagagctgaagatctacttctggctaataaaagatcagagctctagtgatctgtgtgttggttttttgtgtctgcattctagctctagtgatcagcagttcaacctgttgatagtatgtactaagctctcatgtttaatgtactaagctctcatgtttaatgaactaaaccctcatggctaatgtactaagctctcatggctaatgtactaagctctcatgtttcatgtactaagctctcatgtttgaacaataaaattaatataaatcagcaacttaaatagcctctaaggttttaagttttataagaaaaaaaagaatatataaggcttttaaaggttttaaggtttcctaggttatcctcatatgagctcttagaaaaactcatccagcatcaaatgaaactgcaatttattcatatcaggattatcaataccatatttttgaaaaagtcttttctgtaatgaaggagaaaactcacccaggcagttccataggatggcaagatcctggtatctgtctgcaattccaactcttccaacatcaatacaacctattaatttcccctcatcaaaaataaggttatcaagtgagaaatcaccatgagtgaccactgaatctggtgagaatggcaaaagattatgcatttctttccagacttgttcaacaggccagccatttctctcatcatcaaaatcactggcatcaaccaaaccattattcattcttgattgggcctgagccagtctaaatactctatcagagttaaaaggacaattacaaacaggaatggaatgcaatcttctcaggaacactgccagggcatcaacaatattttcacctgaatcaggatattcttccaatacctggaatgctgttttccctgggatggcagtggtgagtaaccatgcatcatcaggagttctgataaaatgcttgatggttggaagaggcataaattcagtcagccagtttagtctgaccatctcatctgtaacatcattggcaacagaacctttgccatgtttcagaaacaactctggggcatctggcttcccatacaatctatagattgtggcacctgattgcccaacattatctctagcccatttatacccatataaatcagcatccatgttggaatttaatcttggcctggagcaagaggtttctctttgaatatggctcatacatgtgcacctcctatagtgagttgtattatactatgcagatatactatgccaatgtttaattgtcag

SEQ ID NO: 6 - DNA, 5131 bp, synthetic construct
  source 1..5131: mol_type = other DNA; note = synthetic
cactatgtggacatgaattcaattggctagcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttggtaagtcactgactgtctatgcctgggaaagggtgggcaggagatggggcagtgcaggaaaagtggcactatgaaccctgcagccctagacaattgtactaaccttcttctctttcctctcctgacaggttggtaaccaagctttccatgggttggagcctcatcttgctcttccttgtagctgttgctactagagtcctgtcccaggtacaactgcagcagcctggggctgagctggtgaagcctggggcctcagtgaagatgtcctgcaaggcttctggctacacatttaccagttacaatatgcactgggtaaaacagacacctggtaggggcctggaatggattggagctatttatcctggaaatggtgatacttcctacaatcagaagttcaaaggcaaggccacattgactgcagacaaatcctccagcacagcctacatgcagctcagcagcctgacatctgaggactctgcagtctattactgtgcaagatcaacttactatggtggtgactggtacttcaatgtctggggtgcagggaccacagtcacagtctctgcagcaagcaccaagggcccatctgtcttccccctggcaccctcctccaagagcacctctgggggcacagctgccctgggctgcctggtcaaggactacttccctgaacctgtgacagtgtcatggaactcaggagccctgaccagtggtgtgcacaccttccctgctgtcctacagtcctcaggactctactccctcagcagtgtggtgactgtgccctccagcagcttgggcacccagacctacatctgcaatgtgaatcacaagcccagcaacaccaaggtggacaagaaagcagagcccaaatcttgtgacaaaactcacacatgcccaccttgcccagcacctgaactcctggggggaccttcagtcttcctcttccccccaaaacccaaggacaccctcatgatctccaggacccctgaggtcacatgtgtggtggtggatgtgagccatgaagaccctgaggtcaagttcaactggtatgtggatggtgtggaggtgcataatgccaagacaaagccaagggaggagcagtacaacagcacttacagagtggtcagtgtcctcactgtcctgcaccaggactggctgaatggcaaggagtacaagtgcaaggtctccaacaaagccctcccagcccccatagagaaaaccatctccaaagccaaagggcagcccagagaaccacaggtgtacaccctgcccccatccagggatgagctgaccaagaaccaggtcagcctgacctgcctggtcaaaggcttctatcccagtgacattgctgtggagtgggagagcaatgggcagcctgagaacaactacaagaccactcctcctgtgctggactctgatggctccttcttcctctacagcaagctcacagtggacaagagcaggtggcagcaggggaatgtcttctcatgctcagtcatgcatgaggctctgcacaaccactacacacagaagagcctctccctgtctccaggtaaatgatagatctacttctggctaataaaagatcagagctctagtgatctgtgtgttggttttttgtgtctgcattctagcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttggtaagtcactgactgtctatgcctgggaaagggtgggcaggagatggggcagtgcaggaaaagtggcactatgaaccctgcagccctagacaattgtactaaccttcttctctttcctctcctgacaggttggtaaccaagctttccatggattttcaggtgcagattatcagcttcctcctaatcagtgcttcagtcataatgtccagaggacaaattgttctctcccagtctccagcaatcctgtctgcatctccaggggagaaggtcacaatgacttgcagggccagctcaagtgtaagttacatccactggttccagcagaagccaggatcctcccccaaaccctggatttatgccacatccaacctggcttctggagtccctgttagattcagtggcagtgggtctgggacttcttactctctcaccatcagcagagtggaggctgaagatgctgccacttattactgccagcagtggactagtaacccacccacttttggaggggggaccaagctggaaatcaaaagaacagtggctgcaccatctgtcttcatcttccctccatctgatgagcagttgaaatctggaactgcctctgttgtgtgcctgctgaataacttctatcccagagaggccaaagtacagtggaaggtggataatgccctccaatcaggtaactcccaggagagtgtcacagagcaggacagcaaggacagcacctacagcctcagcagcaccctgacactgagcaaagcagactatgagaaacacaaagtctatgcctgtgaagtcacccatcagggcctgagctctcctgtcacaaagagcttcaacaggggagagtcttgatagatctacttctggctaataaaagatcagagctctagtgatctgtgtgttggttttttgtgtctgcattctagctctagtgatcagcagttcaacctgttgatagtatgtactaagctctcatgtttaatgtactaagctctcatgtttaatgaactaaaccctcatggctaatgtactaagctctcatggctaatgtactaagctctcatgtttcatgtactaagctctcatgtttgaacaataaaattaatataaatcagcaacttaaatagcctctaaggttttaagttttataagaaaaaaaagaatatataaggcttttaaaggttttaaggtttcctaggttatcctcatatgagctcttagaaaaactcatccagcatcaaatgaaactgcaatttattcatatcaggattatcaataccatatttttgaaaaagtcttttctgtaatgaaggagaaaactcacccaggcagttccataggatggcaagatcctggtatctgtctgcaattccaactcttccaacatcaatacaacctattaatttcccctcatcaaaaataaggttatcaagtgagaaatcaccatgagtgaccactgaatctggtgagaatggcaaaagattatgcatttctttccagacttgttcaacaggccagccatttctctcatcatcaaaatcactggcatcaaccaaaccattattcattcttgattgggcctgagccagtctaaatactctatcagagttaaaaggacaattacaaacaggaatggaatgcaatcttctcaggaacactgccagggcatcaacaatattttcacctgaatcaggatattcttccaatacctggaatgctgttttccctgggatggcagtggtgagtaaccatgcatcatcaggagttctgataaaatgcttgatggttggaagaggcataaattcagtcagccagtttagtctgaccatctcatctgtaacatcattggcaacagaacctttgccatgtttcagaaacaactctggggcatctggcttcccatacaatctatagattgtggcacctgattgcccaacattatctctagcccatttatacccatataaatcagcatccatgttggaatttaatcttggcctggagcaagaggtttctctttgaatatggctcatacatgtgcacctcctatagtgagttgtattatactatgcagatatactatgccaatgtttaattgtcag

SEQ ID NO: 7 - DNA, 5147 bp, synthetic construct
  source 1..5147: mol_type = other DNA; note = synthetic
cactatgtggacatgaattcaattggctagcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttggtaagtcactgactgtctatgcctgggaaagggtgggcaggagatggggcagtgcaggaaaagtggcactatgaaccctgcagccctagacaattgtactaaccttcttctctttcctctcctgacaggttggtaacctgacaggttggtaaccaagctttccatgggttggagcctcatcttgctcttccttgtcgctgttgctacgcgtgtcctgtcccaggtacaactgcagcagcctggggctgagctggtgaagcctggggcctcagtgaagatgtcctgcaaggcttctggctacacatttaccagttacaatatgcactgggtaaaacagacacctggtcggggcctggaatggattggagctatttatcccggaaatggtgatacttcctacaatcagaagttcaaaggcaaggccacattgactgcagacaaatcctccagcacagcctacatgcagctcagcagcctgacatctgaggactctgcggtctattactgtgcaagatcgacttactacggcggtgactggtacttcaatgtctggggcgcagggaccacggtcaccgtctctgcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagcagagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtcatgcatgaggctctgcacaaccactacacgcagaagagcctctccctgtctccgggtaaatgatagatctacttctggctaataaaagatcagagctctagtgatctgtgtgttggttttttgtgtctgcattctagcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttggtaagtcactgactgtctatgcctgggaaagggtgggcaggagatggggcagtgcaggaaaagtggcactatgaaccctgcagccctagacaattgtactaaccttcttctctttcctctcctgacaggttggtaaccaagctttccatggattttcaggtgcagattatcagcttcctcctaatcagtgcttcagtcataatgtccagaggacaaattgttctctcccagtctccagcaatcctgtctgcatctccaggggagaaggtcacaatgacttgcagggccagctcaagtgtaagttacatccactggttccagcagaagccaggatcctcccccaaaccctggatttatgccacatccaacctggcttctggagtccctgttcgcttcagtggcagtgggtctgggacttcttactctctcaccatcagcagagtggaggctgaagatgctgccacttattactgccagcagtggactagtaacccacccacgttcggaggggggaccaagctggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtcttgatagatctacttctggctaataaaagatcagagctctagtgatctgtgtgttggttttttgtgtctgcattctagctctagtgatcagcagttcaacctgttgatagtatgtactaagctctcatgtttaatgtactaagctctcatgtttaatgaactaaaccctcatggctaatgtactaagctctcatggctaatgtactaagctctcatgtttcatgtactaagctctcatgtttgaacaataaaattaatataaatcagcaacttaaatagcctctaaggttttaagttttataagaaaaaaaagaatatataaggcttttaaaggttttaaggtttcctaggttatcctcatatgagctcttagaaaaactcatccagcatcaaatgaaactgcaatttattcatatcaggattatcaataccatatttttgaaaaagtcttttctgtaatgaaggagaaaactcacccaggcagttccataggatggcaagatcctggtatctgtctgcaattccaactcttccaacatcaatacaacctattaatttcccctcatcaaaaataaggttatcaagtgagaaatcaccatgagtgaccactgaatctggtgagaatggcaaaagattatgcatttctttccagacttgttcaacaggccagccatttctctcatcatcaaaatcactggcatcaaccaaaccattattcattcttgattgggcctgagccagtctaaatactctatcagagttaaaaggacaattacaaacaggaatggaatgcaatcttctcaggaacactgccagggcatcaacaatattttcacctgaatcaggatattcttccaatacctggaatgctgttttccctgggatggcagtggtgagtaaccatgcatcatcaggagttctgataaaatgcttgatggttggaagaggcataaattcagtcagccagtttagtctgaccatctcatctgtaacatcattggcaacagaacctttgccatgtttcagaaacaactctggggcatctggcttcccatacaatctatagattgtggcacctgattgcccaacattatctctagcccatttatacccatataaatcagcatccatgttggaatttaatcttggcctggagcaagaggtttctctttgaatatggctcatacatgtgcacctcctatagtgagttgtattatactatgcagatatactatgccaatgtttaattgtcag

SEQ ID NO: 8 - DNA, 5128 bp, synthetic construct
  source 1..5128: mol_type = other DNA; note = synthetic
cactatgtggacatgaattcaattggctagcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttggtaagtcactgactgtctatgcctgggaaagggtgggcaggagatggggcagtgcaggaaaagtggcactatgaaccctgcagccctagacaattgtactaaccttcttctctttcctctcctgacaggttggtaaccaagctttccatgggctggtccctgatcctgctgttcctggtggctgtggccaccagagtgctgagccaggtgcagctgcagcagcctggggctgagcttgtgaaacctggggcctctgtgaagatgagctgcaaggcctctggctacaccttcaccagctacaacatgcactgggtcaagcagacccctggcagaggcctggaatggattggagccatctaccctggcaatggggacacctcctacaaccagaagttcaagggcaaggccaccctgacagctgacaagagcagcagcacagcctacatgcagctgtccagcctgacctctgaggactctgctgtgtactactgtgccaggtccacctactatgggggagactggtacttcaatgtgtggggagctggcaccacagtgacagtgtctgctgccagcaccaagggcccctctgtgtttcctctggcccccagcagcaagagcacctctgggggaacagctgccctgggctgccttgtgaaggactacttccctgagcctgtgactgtgtcctggaactctggggccctgacatctggggtgcacaccttccctgcagtgctgcagtccagtggcctgtactccctgtcctctgttgtgacagtgcccagctccagcctgggcacccagacctacatctgcaatgtgaaccacaagcccagcaacaccaaggtggacaagaaggctgagcccaagagctgtgacaagacccacacctgtcccccctgtcctgcccctgaactgctgggaggaccttctgtgttcctgttcccacccaagcccaaggataccctgatgatcagcagaacccctgaagtgacctgtgtggtggtggatgtgtcccatgaggacccagaagtgaagttcaattggtatgtggatggggtggaagtgcacaatgccaagaccaagcccagagaggaacagtacaacagcacctacagagtggtgtctgtgctgactgtgctgcaccaggactggctgaatggcaaagagtacaagtgcaaggtgtccaacaaggccctgccagcccccattgagaaaaccatcagcaaggccaagggccagcctagagaaccccaggtgtacacactgccccctagcagggatgagctgaccaagaaccaggtgtccctgacatgccttgtgaaaggcttctacccctctgacattgctgtggaatgggagagcaatggacagcctgagaacaactacaagaccaccccccctgtgctggactctgatggctcattcttcctgtacagcaagctgacagtggacaagtccagatggcagcagggcaatgtgttcagctgctctgtgatgcatgaggccctgcacaaccactacacccagaaaagcctgtccctgtcccctggcaagtgaagatctacttctggctaataaaagatcagagctctagtgatctgtgtgttggttttttgtgtctgcattctagcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttggtaagtcactgactgtctatgcctgggaaagggtgggcaggagatggggcagtgcaggaaaagtggcactatgaaccctgcagccctagacaattgtactaaccttcttctctttcctctcctgacaggttggtaaccaagccaccatggacttccaggtgcagatcatcagctttctgctgatctctgcctctgtgatcatgagcagaggccagattgtgctgagccagagccctgccatcctgtctgcaagccctggggagaaagtgaccatgacctgcagagccagcagctctgtgtcctacatccactggttccagcagaagcctggcagcagccccaagccttggatctatgccaccagcaacctggcatctggggtgccagtcagattctctggctctggatctggcaccagctacagcctgaccatcagcagagtggaagctgaggatgctgccacctactactgccagcagtggaccagcaatccccccacctttggagggggcaccaagctggaaatcaagagaacagtggctgccccctctgtgttcatcttcccaccctctgatgagcagctgaagtctggaacagcctctgttgtgtgcctgctgaacaacttctaccccagagaagccaaggtgcagtggaaggtggacaatgccctgcagtctggcaactcccaggaatctgtgacagagcaggacagcaaggactccacctactccctgagcagcaccctgaccctgagcaaggctgactatgagaagcacaaagtgtatgcctgtgaagtgacccaccagggcctgtccagccctgtgaccaagagcttcaacagaggggagagctgaagatctacttctggctaataaaagatcagagctctagtgatctgtgtgttggttttttgtgtctgcattctagctctagtgatcagcagttcaacctgttgatagtatgtactaagctctcatgtttaatgtactaagctctcatgtttaatgaactaaaccctcatggctaatgtactaagctctcatggctaatgtactaagctctcatgtttcatgtactaagctctcatgtttgaacaataaaattaatataaatcagcaacttaaatagcctctaaggttttaagttttataagaaaaaaaagaatatataaggcttttaaaggttttaaggtttcctaggttatcctcatatgagctcttagaaaaactcatccagcatcaaatgaaactgcaatttattcatatcaggattatcaataccatatttttgaaaaagtcttttctgtaatgaaggagaaaactcacccaggcagttccataggatggcaagatcctggtatctgtctgcaattccaactcttccaacatcaatacaacctattaatttcccctcatcaaaaataaggttatcaagtgagaaatcaccatgagtgaccactgaatctggtgagaatggcaaaagattatgcatttctttccagacttgttcaacaggccagccatttctctcatcatcaaaatcactggcatcaaccaaaccattattcattcttgattgggcctgagccagtctaaatactctatcagagttaaaaggacaattacaaacaggaatggaatgcaatcttctcaggaacactgccagggcatcaacaatattttcacctgaatcaggatattcttccaatacctggaatgctgttttccctgggatggcagtggtgagtaaccatgcatcatcaggagttctgataaaatgcttgatggttggaagaggcataaattcagtcagccagtttagtctgaccatctcatctgtaacatcattggcaacagaacctttgccatgtttcagaaacaactctggggcatctggcttcccatacaatctatagattgtggcacctgattgcccaacattatctctagcccatttatacccatataaatcagcatccatgttggaatttaatcttggcctggagcaagaggtttctctttgaatatggctcatacatgtgcacctcctatagtgagttgtattatactatgcagatatactatgccaatgtttaattgtcag

SEQ ID NO: 9 - DNA, 7413 bp, synthetic construct
  source 1..7413: mol_type = other DNA; note = synthetic
cactatgtgttaattaaaattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaaggaatttgaattctcatagctagcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttggtaagtcactgactgtctatgcctgggaaagggtgggcaggagatggggcagtgcaggaaaagtggcactatgaaccctgcagccctagacaattgtactaaccttcttctctttcctctcctgacaggttggtaaccaagccaccatgggctggtccctgatcctgctgttcctggtggctgtggccaccagagtgctgagccaggtgcagctgcagcagcctggggctgagcttgtgaaacctggggcctctgtgaagatgagctgcaaggcctctggctacaccttcaccagctacaacatgcactgggtcaagcagacccctggcagaggcctggaatggattggagccatctaccctggcaatggggacacctcctacaaccagaagttcaagggcaaggccaccctgacagctgacaagagcagcagcacagcctacatgcagctgtccagcctgacctctgaggactctgctgtgtactactgtgccaggtccacctactatgggggagactggtacttcaatgtgtggggagctggcaccacagtgacagtgtctgctgccagcaccaagggcccctctgtgtttcctctggcccccagcagcaagagcacctctgggggaacagctgccctgggctgccttgtgaaggactacttccctgagcctgtgactgtgtcctggaactctggggccctgacatctggggtgcacaccttccctgcagtgctgcagtccagtggcctgtactccctgtcctctgttgtgacagtgcccagctccagcctgggcacccagacctacatctgcaatgtgaaccacaagcccagcaacaccaaggtggacaagaaggctgagcccaagagctgtgacaagacccacacctgtcccccctgtcctgcccctgaactgctgggaggaccttctgtgttcctgttcccacccaagcccaaggataccctgatgatcagcagaacccctgaagtgacctgtgtggtggtggatgtgtcccatgaggacccagaagtgaagttcaattggtatgtggatggggtggaagtgcacaatgccaagaccaagcccagagaggaacagtacaacagcacctacagagtggtgtctgtgctgactgtgctgcaccaggactggctgaatggcaaagagtacaagtgcaaggtgtccaacaaggccctgccagcccccattgagaaaaccatcagcaaggccaagggccagcctagagaaccccaggtgtacacactgccccctagcagggatgagctgaccaagaaccaggtgtccctgacatgccttgtgaaaggcttctacccctctgacattgctgtggaatgggagagcaatggacagcctgagaacaactacaagaccaccccccctgtgctggactctgatggctcattcttcctgtacagcaagctgacagtggacaagtccagatggcagcagggcaatgtgttcagctgctctgtgatgcatgaggccctgcacaaccactacacccagaaaagcctgtccctgtcccctggcaagtgaagatctacttctggctaataaaagatcagagctctagtgatctgtgtgttggttttttgtgtctgcattctagcagagccccactgtgttcatcttacagatggaaatactgacattcagaggagttagttaacttgcctaggtgattcagctaataagtgcaagaaagatttcaatccaaggtgatttgattctgaagcctgtgctaatcacattacaccaagctacaacttcatttataaataataagtcagctttcaagggcctttcaggtgtcctgcacttctacaagctgtgccatttagtgaacacaaaatgagccttctgatgaagtagtcttttcattatttcagatattagaacactaaaattcttagctgccagctgattgaaggctgggacaaaattcaaacatgcatctacaacaatatatatctcaatgttagtctccaaattctattgacttcaactcaagagaatataaagagctagtctttatacactctttaaggtatgatatcatctggaaagtaacaaaattgatgcaaatttgaatgaactttatcatggtgtatttacacaatgtgtttcttctccctgcaatgtatttctttctctaattccttccatttgatctttcatacacaatctggttctgatgtatgttttttggatgcacttttcaactccaaaagacagagctagttactttcttcctggtgctccaagcactgtatttgtatctgtattcaagccctttgcaatattgtactggatcattatttcacctctaggatggcttccccaggcaacttgtgttcacccagagactacattttgtatcttgttgacctttgaacttccaccagtgtctaaaaataatatgtatgcaaaattacttgctatgagaatgtataattaaacaatataaaaaggagaagcaaggagagaaacacaggtgtgtatttgtgtttgtgtgcttaaaaggcagtgtggaaaaggaagaaatgccatttatagtgaggagacaaagttatattacctcttatctggcttttaaggagattttgctgagctaaaaatcctatattcatagaaaagccttacctgagttgccaatacctcaattcagtctagcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttggtaagtcactgactgtctatgcctgggaaagggtgggcaggagatggggcagtgcaggaaaagtggcactatgaaccctgcagccctagacaattgtactaaccttcttctctttcctctcctgacaggttggtaaccaagccaccatggacttccaggtgcagatcatcagctttctgctgatctctgcctctgtgatcatgagcagaggccagattgtgctgagccagagccctgccatcctgtctgcaagccctggggagaaagtgaccatgacctgcagagccagcagctctgtgtcctacatccactggttccagcagaagcctggcagcagccccaagccttggatctatgccaccagcaacctggcatctggggtgccagtcagattctctggctctggatctggcaccagctacagcctgaccatcagcagagtggaagctgaggatgctgccacctactactgccagcagtggaccagcaatccccccacctttggagggggcaccaagctggaaatcaagagaacagtggctgccccctctgtgttcatcttcccaccctctgatgagcagctgaagtctggaacagcctctgttgtgtgcctgctgaacaacttctaccccagagaagccaaggtgcagtggaaggtggacaatgccctgcagtctggcaactcccaggaatctgtgacagagcaggacagcaaggactccacctactccctgagcagcaccctgaccctgagcaaggctgactatgagaagcacaaagtgtatgcctgtgaagtgacccaccagggcctgtccagccctgtgaccaagagcttcaacagaggggagagctgaagatctacttctggctaataaaagatcagagctctagtgatctgtgtgttggttttttgtgtctgcattctagcagtcaatatgttcaccccaaaaaagctgtttgttaacttgtcaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggctctagctctagtgatcagcagttcaacctgttgatagtatgtactaagctctcatgtttaatgtactaagctctcatgtttaatgaactaaaccctcatggctaatgtactaagctctcatggctaatgtactaagctctcatgtttcatgtactaagctctcatgtttgaacaataaaattaatataaatcagcaacttaaatagcctctaaggttttaagttttataagaaaaaaaagaatatataaggcttttaaaggttttaaggtttcctaggttatcctcatatgagctcttagaaaaactcatccagcatcaaatgaaactgcaatttattcatatcaggattatcaataccatatttttgaaaaagtcttttctgtaatgaaggagaaaactcacccaggcagttccataggatggcaagatcctggtatctgtctgcaattccaactcttccaacatcaatacaacctattaatttcccctcatcaaaaataaggttatcaagtgagaaatcaccatgagtgaccactgaatctggtgagaatggcaaaagattatgcatttctttccagacttgttcaacaggccagccatttctctcatcatcaaaatcactggcatcaaccaaaccattattcattcttgattgggcctgagccagtctaaatactctatcagagttaaaaggacaattacaaacaggaatggaatgcaatcttctcaggaacactgccagggcatcaacaatattttcacctgaatcaggatattcttccaatacctggaatgctgttttccctgggatggcagtggtgagtaaccatgcatcatcaggagttctgataaaatgcttgatggttggaagaggcataaattcagtcagccagtttagtctgaccatctcatctgtaacatcattggcaacagaacctttgccatgtttcagaaacaactctggggcatctggcttcccatacaatctatagattgtggcacctgattgcccaacattatctctagcccatttatacccatataaatcagcatccatgttggaatttaatcttggcctggagcaagaggtttctctttgaatatggctcatacatgtgcacctcctatagtgagttgtattatactatgcagatatactatgccaatgtttaattgtcag

SEQ ID NO: 10 - DNA, 3492 bp, synthetic construct
  source 1..3492: mol_type = other DNA; note = synthetic
cactatgtggacatgaattcaattggctagcaggagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttggtaagtcactgactgtctatgcctgggaaagggtgggcaggagatggggcagtgcaggaaaagtggcactatgaaccctgcagccctagacaattgtactaaccttcttctctttcctctcctgacaggttggtaaccaagccaccatgcagagagtgaatatgatcatggctgagagccctggcctgatcaccatctgcctgctgggctacctgctgtctgctgagtgcacagtgtttctggaccatgagaatgccaacaagatcctgaacaggcccaagaggtacaactctggcaagctggaagagtttgtgcagggcaacctggaaagggaatgcatggaagagaagtgcagctttgaagaggccagggaagtgtttgagaacacagagagaaccacagagttctggaagcagtatgtggatggggaccagtgtgaaagcaacccctgcctgaatgggggcagctgcaaggatgacatcaacagctatgagtgctggtgcccctttggctttgagggcaagaactgtgaactggatgtgacctgcaacatcaagaatggcagatgtgaacagttctgcaagaactctgctgacaacaaggttgtgtgctcctgcacagagggctacagactggctgagaaccagaaaagctgtgaacctgctgtgcccttcccatgtggcagagtgtctgtgtcccagaccagcaagctgaccagagctgagacagtgttccctgatgtggactatgtgaactccacagaggctgaaaccatcctggacaacatcacccagagcacccagtccttcaatgacttcaccagagttgtgggaggggaggatgccaagcctggccagttcccatggcaagtggtgctgaatggcaaagtggatgccttctgtgggggctccattgtgaatgagaagtggattgtgacagctgcccactgtgtggaaactggagtgaagatcacagtggtggctggggagcacaacattgaggaaacagagcacacagagcagaaaagaaatgtgatcaggatcatcccccaccacaactacaatgctgccatcaacaagtacaaccatgacattgccctgctggaactggatgagcccctggtgctgaacagctatgtgacccccatctgcattgctgacaaagagtacaccaacatctttctgaagtttggctctggctatgtgtctggctggggcagggtgttccacaagggaaggagtgctctggtgctgcagtacctgagagtgccactggtggacagagccacctgtctgagaagcaccaagttcaccatctacaacaacatgttctgtgctggcttccatgaggggggcagagactcctgccagggggattctgggggccctcatgtgacagaggtggaaggcaccagctttctgacaggcatcatcagctggggagaggaatgtgccatgaagggcaaatatggcatctacaccaaggtgtccagatatgtgaattggatcaaagaaaagaccaagctgacatgaagatctacttctggctaataaaagatcagagctctagtgatctgtgtgttggttttttgtgtctgcattctagctctagtgatcagcagttcaacctgttgatagtatgtactaagctctcatgtttaatgtactaagctctcatgtttaatgaactaaaccctcatggctaatgtactaagctctcatggctaatgtactaagctctcatgtttcatgtactaagctctcatgtttgaacaataaaattaatataaatcagcaacttaaatagcctctaaggttttaagttttataagaaaaaaaagaatatataaggcttttaaaggttttaaggtttcctaggttatcctcatatgagctcttagaaaaactcatccagcatcaaatgaaactgcaatttattcatatcaggattatcaataccatatttttgaaaaagtcttttctgtaatgaaggagaaaactcacccaggcagttccataggatggcaagatcctggtatctgtctgcaattccaactcttccaacatcaatacaacctattaatttcccctcatcaaaaataaggttatcaagtgagaaatcaccatgagtgaccactgaatctggtgagaatggcaaaagattatgcatttctttccagacttgttcaacaggccagccatttctctcatcatcaaaatcactggcatcaaccaaaccattattcattcttgattgggcctgagccagtctaaatactctatcagagttaaaaggacaattacaaacaggaatggaatgcaatcttctcaggaacactgccagggcatcaacaatattttcacctgaatcaggatattcttccaatacctggaatgctgttttccctgggatggcagtggtgagtaaccatgcatcatcaggagttctgataaaatgcttgatggttggaagaggcataaattcagtcagccagtttagtctgaccatctcatctgtaacatcattggcaacagaacctttgccatgtttcagaaacaactctggggcatctggcttcccatacaatctatagattgtggcacctgattgcccaacattatctctagcccatttatacccatataaatcagcatccatgttggaatttaatcttggcctggagcaagaggtttctctttgaatatggctcatacatgtgcacctcctatagtgagttgtattatactatgcagatatactatgccaatgtttaattgtcag

(19) United States
(12) Patent Application Publication
(10) Pub. No.: US 2024/0350662 A1
(43) Pub. Date: Oct. 24, 2024

(54) SYSTEMS AND METHODS FOR NUCLEIC ACID EXPRESSION IN VIVO

(71) Applicant: DNARx, San Francisco, CA (US)

(72) Inventors: Robert James DEBS, San Francisco, CA (US); Timothy D. HEATH, Madison, WI (US); Chakkrapong HANDUMRONGKUL, Richmond, VA (US)

(73) Assignee: DNARx, San Francisco, CA (US)

(21) Appl. No.: 18/512,804

(22) Filed: Nov. 17, 2023

Related U.S. Application Data
(62) Division of application No. 16/140,086, filed on Sep. 24, 2018, now abandoned.
Application No. 16/140,086 is a division of application No. 15/268,000, filed on Sep. 16, 2016, now Pat. No. 10,086,089.
(60) Provisional application No. 62/220,646, filed on Sep. 18, 2015.

(51) Int. Cl. A61K 48/00; A61K 9/127; A61K 31/19; A61K 31/522; A61K 31/573; A61K 38/19; A61K 38/48; A61K 39/395; A61K 47/69; C07K 16/00; C07K 16/28; A61K 31/00; A61K 39/00; C07H 21/04; C12N 15/85

(52) U.S. Cl. CPC A61K 48/0016; A61K 9/1272; A61K 31/19; A61K 31/522; A61K 31/573; A61K 38/193; A61K 38/4846; A61K 39/395; A61K 47/6911; A61K 48/0066; A61K 48/0083; C07K 16/00; C07K 16/2887; C12Y 304/21022; A61K 31/00; A61K 2039/505; A61K 2039/53; A61K 48/005; C07H 21/04; C07K 2317/24; C12N 15/85

(57) ABSTRACT

The present invention provides compositions, systems, kits, and methods for generating expression of one or more proteins and/or biologically active nucleic acid molecules in a subject (e.g., at therapeutic levels for extended periods required to produce therapeutic effects). In certain embodiments, systems and kits are provided that comprise a first composition comprising a first amount of polycationic structures, and a second composition comprising a therapeutically effective amount of expression vectors (e.g., non-viral expression vectors not associated with liposomes) that are CpG-free or CpG-reduced, where the expression vectors comprise a first nucleic acid sequence encoding: i) a first therapeutic protein or proteins, and/or ii) a first biologically active nucleic acid molecule or molecules.

This present application is a divisional of U.S. patent application Ser. No. 16/140,086, filed Sep. 24, 2018, now abandoned, which is a divisional of U.S. patent application Ser. No. 15/268,000, filed Sep. 16, 2016, now U.S. Pat. No. 10,086,089, issued Oct. 2, 2018, which claims the benefit of U.S. Provisional Application No. 62/220,646, filed Sep. 18, 2015, the disclosure of each of which is incorporated by reference herein in its entirety.

This application contains a computer readable Sequence Listing which has been submitted in XML file format via Patent Center, the entire content of which is incorporated by reference herein in its entirety. The Sequence Listing XML file submitted via Patent Center is entitled “14755-010-999_SEQ_LISTING.xml”, was created on Nov. 13, 2023, and is 40,795 bytes in size

### FIELD OF THE INVENTION

The present invention provides compositions, systems, kits, and methods for expression of one or more proteins or biologically active nucleic acid molecules in a subject, human or non-human mammal, (e.g., at therapeutic levels for the extended periods of time required to produce therapeutic effects). In certain embodiments, systems and kits are provided that comprise a first composition comprising polycationic structures (e.g., empty cationic liposomes, cationic micelles, cationic emulsions, or cationic polymers) and a second composition comprising expression vectors (e.g., non-viral expression vectors not associated with liposomes or other carriers) that are CpG-free or CpG-reduced, that comprise a first nucleic acid sequence encoding: i) a first therapeutic protein or proteins, and/or ii) a first biologically active nucleic acid molecule or molecules. In other embodiments, such first and second compositions are sequentially administered (e.g., systemically) to a subject such that the therapeutic protein and/or the biologically active nucleic acid molecule is/are expressed in the subject (e.g., at a therapeutic level, for at least 5 or at least 50 days, such that a disease or condition is treated or a physiological or disease trait is altered).

### BACKGROUND

The simplest non-viral gene delivery system uses naked expression vector DNA. Direct injection of free DNA into certain tissues, particularly muscle, has been shown to produce high levels of gene expression, and the simplicity of this approach has led to its adoption in a number of clinical protocols. In particular, this approach has been applied to the gene therapy of cancer where the DNA can be injected either directly into the tumor or can be injected into muscle cells in order to express tumor antigens that might function as a cancer vaccine.

Although direct injection of plasmid DNA has been shown to lead to gene expression, the overall level of expression is much lower than with either viral or liposomal vectors. Naked DNA is also generally thought to be unsuitable for systemic administration due to the presence of serum nucleases. As a result, direct injection of plasmid DNA appears to be limited to only a few applications involving tissues that are easily accessible to direct injection such as skin and muscle cells.

### SUMMARY OF THE INVENTION

The present invention provides compositions, systems, kits, and methods for expression of a protein or proteins and/or biologically active nucleic acid molecule(s) in a subject (e.g., at therapeutic levels for the extended periods of time required to produce therapeutic effects in the host). In certain embodiments, systems and kits are provided that comprise a first composition comprising a first amount of polycationic structures (e.g., empty cationic liposomes, empty cationic micelles, or empty cationic emulsions), and a second composition comprising a therapeutically effective amount of expression vector(s) (e.g., non-viral expression vectors not associated with liposomes) that are CpG-free or CpG-reduced, where the expression vectors comprise a first nucleic acid sequence encoding: i) a first therapeutic protein (or non-therapeutic protein, such as a marker protein), and/or ii) a first biologically active nucleic acid molecule. In certain embodiments, the expression vector comprises a second, third, or fourth nucleic acid sequence encoding a second, third, and/or fourth therapeutic or non-therapeutic protein, and/or a second, third, or fourth biologically active nucleic acid molecule. In some embodiments, the first nucleic acid sequences further encode a second, third, fourth, fifth, and/or sixth therapeutic protein, and/or a second, third, fourth, fifth, and/or sixth biologically active nucleic acid molecule. In other embodiments, such first and second compositions are sequentially administered (e.g., systemically) to a subject such that the therapeutic protein(s) and/or the biologically active nucleic acid molecule(s) is/are expressed in the subject (e.g., at a therapeutic level, for at least 5 or at least 50 days, or at least 100 . . . 200 . . . or at least 400 days, such that disease(s) or condition(s) is/are treated or physiological trait(s) is/are altered).

In some embodiments, provided herein are methods of expressing a first therapeutic protein and/or a biologically active nucleic acid molecule in a subject (e.g., human or non-human mammal) comprising: a) administering (e.g., systemically) a first composition to a subject, wherein the first composition comprises first amount of polycationic structures (e.g., empty cationic liposomes, empty cationic micelles, or empty cationic emulsions) and wherein the first composition is free, or essentially free, of nucleic acid molecules (e.g., nucleic acid is un-detectable or barely detectable in the composition); and b) administering (e.g., systemically, intravascularly, etc.) a second composition to the subject (e.g., initiating within about 2 . . . 10 . . . 50 . . . 100 . . . 200 . . . 300 . . . 400 minutes of administering the first composition), wherein the second composition comprises an amount of expression vectors (e.g., non-viral expression vectors not associated with liposomes or any other carrier), wherein the expression vectors are CpG-free or CpG-reduced, wherein each of the expression vectors comprise nucleic acid sequence(s) encoding: i) first, second, third, fourth, fifth, and/or sixth therapeutic protein(s) or non-therapeutic, and/or ii) first, second, third, fourth, fifth, and/or sixth biologically active nucleic acid molecule(s). In certain embodiments, as a result of the administering the first composition and the administering the second composition, the first therapeutic or non-therapeutic protein and/or the biologically active nucleic acid molecule is/are expressed in the subject (e.g., at a therapeutic level, for at least 5 . . . 50 . . . 100 . . . 300 days . . . 400 days or longer, with respect to a disease or condition, or at an effective level sufficient to alter a physiological or disease trait). In certain embodiments, the polycationic structures (e.g., empty liposomes) present in the first composition have a z-average diameter of about 20-85 nm (e.g., 20 . . . 25 . . . 30 . . . 40 . . . 45 . . . 50 . . . 55 . . . 60 . . . 65 . . . 70 . . . 75 . . . 80 . . . 85 nm). In certain embodiments, the polycationic structures are empty liposomes with a z-average diameter of about 72-76 nm, and are small uni-lammellar vesicles.

In some embodiments, provided herein are methods of expressing a first therapeutic or non-therapeutic protein and/or a biologically active nucleic acid molecule in a subject comprising: a) administering a first composition to a subject, wherein the subject has at least one symptom of a disease or condition, or has at least physiological trait to be altered, wherein the first composition comprises a first amount of polycationic structures (e.g., empty cationic liposomes, empty cationic micelles, or empty cationic emulsions), and wherein the first composition is free, or essentially free, of nucleic acid molecules; and b) administering (or initiating administration of) a second composition to the subject within about 100 minutes or about 200 . . . or 400 minutes of administering said first composition, wherein the second composition comprises a therapeutically effective amount of expression vectors, wherein the expression vectors are CpG-free or CpG-reduced, wherein the expression vectors each comprise a first nucleic acid sequence encoding: i) a first therapeutic or non-therapeutic protein, and/or ii) a first biologically active nucleic acid molecule, c) administering dexamethasone palmitate and/or neutral lipids to the subject, either in said first and/or second composition, or present in a third composition (e.g., within 100 or 200 . . . or 400 minutes of administration of the first or second compositions). In some embodiments, as a result of the administering the first composition, the administering the second composition, and the administering of the dexamethasone palmitate and/or neutral lipids, the first therapeutic protein and/or the biologically active nucleic acid molecule is/are expressed in the subject at a therapeutic level with respect to the disease or condition, or at an effective level sufficient to alter the physiological or disease trait.

In certain embodiments, dexamethasone palmitate is in the first composition, and wherein 2.0% to 6.0% (e.g., 2.0% . . . 2.5% . . . 3.0%) of the first composition comprises the dexamethasone palmitate. In certain embodiments, the dexamethasone palmitate is administered in the third composition, which is administered before the first and/or second composition is administered, or is administered after the first and/or second composition, but within 100 . . . 400 minutes thereof. In certain embodiments, the methods further comprise d) administering dexamethasone to the subject, either in the first and/or second and/or third composition, or present in a fourth composition (e.g., initiating within 100 or 300 minutes of administration of the first or second or third compositions, such as before any of the administrations or after the other administrations). In certain embodiments, the polycationic structures (e.g., empty liposomes) present in the first composition have a z-average diameter of about 20-85 nm (e.g., 20 . . . 25 . . . 30 . . . 40 . . . 45 . . . 50 . . . 55 . . . 60 . . . 65 . . . 70 . . . 75 . . . 80 . . . 85 nm). In certain embodiments, the polycationic structures are empty liposomes with a z-average diameter of about 72-76 nm, and are small uni-lammellar vesicles. In some embodiments, A) the ratio is 10:1 to 18:1; B) 2.0% to 6.0% of the first composition comprises dexamethasone or dexamethasone palmitate; and/or C) each of the expression vectors each comprise only a single expression cassette (i.e., no other expression cassettes are present in each vector), wherein the expression cassette comprises the first nucleic acid sequence encoding the first therapeutic protein and a second nucleic acid sequence encoding a second therapeutic protein, and wherein the expression cassette encodes a self-cleaving peptide sequence (or other cleavage sequence) between the first and second nucleic acid sequences. In certain embodiments, the self-cleaving peptide comprises FMDV 2A. In particular embodiments, the first therapeutic protein comprises a monoclonal antibody light chain and the second therapeutic protein comprises a heavy chain of said monoclonal antibody (e.g., the light and heavy chains combine to form an monoclonal antibody fragment (e.g., Fab) or monoclonal antibody when expresses in a subject). In certain embodiments, the polycationic structures comprise empty liposomes. In particular embodiments, the empty liposomes present in said first composition have an average diameter of about 50-85 nm. In certain embodiments, the methods further comprise administering an agent or additional regulating expression vectors, either in said first and/or second composition, or present in a third composition, wherein the agent increases or decreases the expression at the therapeutic or effective level, and/or the length of time of the expression at said therapeutic or effective level, compared to when the drug agent is not administered to said subject (e.g., for therapeutics that need to be expressed for only a certain, limited amount of time). In particular embodiments, the agent is selected from colchicine, dexamethasone, dexamethasone palmitate, neutral lipids, valproic acid, theophylline, sildenafil, amlexanox, chloroquine, SAHA, and L-arginine+sildenafil.

In some embodiments, the expression vectors each further comprise a regulating nucleic acid sequence, wherein the regulating nucleic acid sequence reduces the duration of expression of the first nucleic acid sequence that would occur in the absence of said regulating nucleic acid sequence. In other embodiments, the regulating nucleic acid sequence is selected from the group consisting of: a promoter, an enhancer, a second nucleic acid sequence encoding a second protein, and/or a second biologically active nucleic acid molecule. In additional embodiments, the first amount of polycationic structures in the first composition comprises a mixture of at least a first and second different types of cationic liposomes that reduces the expression of the first therapeutic protein and/or first biologically active nucleic acid molecule compared to such expression when only said first or only said second type of cationic liposomes are employed in said method. In particular embodiments, the therapeutic protein is expressed at a level that is above 1 ug/ml (e.g., 1.1-1.5 ug/ml), and wherein said therapeutic protein is expressed at the level in said subject for at least 7 consecutive days (e.g., at least 7 . . . 21 . . . 50 . . . 100 . . . or 400 days).

In certain embodiments, provided herein are methods of expressing a first therapeutic protein and/or a biologically active nucleic acid molecule in a subject comprising: a) administering (e.g., systemically) a first composition to a subject, wherein the subject has at least one symptom of a disease or condition, or has at least physiological trait to be altered (e.g., level of hematopoietic stem cells), wherein the first composition comprises a first amount of polycationic structures (e.g., empty cationic liposomes, empty cationic micelles, or empty cationic emulsions), and wherein the first composition is free, or essentially free, of nucleic acid molecules; and b) administering (e.g., systemically) a second composition to the subject be initiated (or completed) within about 2 . . . 10 . . . 25 . . . 100 . . . 200 or 400 minutes of administering the first composition, wherein the second composition comprises a therapeutically effective amount of expression vectors (e.g., plasmid), wherein the expression vectors are CpG-free or CpG-reduced (e.g., the nucleic acid sequence of the expression vector has been altered to contain fewer CpG di-nucleotides than normally present in the wild-type version of the sequences in the vector), wherein the expression vectors each comprise nucleic acid sequence(s) encoding: i) a first therapeutic protein (or first and second therapeutic proteins, or first, second, and third therapeutic proteins, etc.), and/or ii) a first biologically active nucleic acid molecule (or first and second or more biologically active nucleic acid molecules), and wherein, as a result of the administering the first composition and the administering the second composition, and wherein, as a result of administering the first and second compositions, the therapeutic protein(s) and/or the biologically active nucleic acid molecule(s) is/are expressed in the subject at a therapeutic level with respect to the disease or condition, or at an effective level sufficient to alter the physiological or disease trait.

In certain embodiments, the expression vectors are not associated with polycationic structures (e.g., empty cationic liposomes, empty cationic micelles, or empty cationic emulsions)), or other molecules, in the second composition (and there are no detectable polycationic structures present in the second composition). In other embodiments, the expression vectors are naked, non-viral, expression vectors (e.g., plasmids). In certain embodiments, the expression vectors are viral expression vectors (e.g., adeno-associated viral vector or adenovirus vector or synthetic mRNA, miRNA, ribozyme or shRNA nucleic acid vectors). In particular embodiments, the first and/or second composition is administered systemically, regionally, transcutaneously, intradermally, orally, intramuscularly, intravenously, into the gastrointestinal tract, bladder or by pulmonary inhalation, or by an intrathecal or intraventricular route.

In certain embodiments, the therapeutic protein or proteins and/or biologically active nucleic acid molecule or molecules is/are expressed at the therapeutic or effective level in the subject on consecutive days for at least 5 . . . 20 . . . 63 . . . 100 . . . 200 . . . 300 days . . . 1 year or more. In some embodiments, the methods further comprise: c) testing the subject (e.g., body imaging or scanning), or a sample (e.g., blood, serum, plasma, tissue, urine, etc.) from the subject, after at least 5 . . . 20 . . . 63 . . . 100 . . . 200 . . . 300 days . . . or 1 year from the administering the first and second compositions, and determining that the therapeutic protein(s) and/or biologically active nucleic acid molecule(s) is/are being expressed in the subject at the therapeutic or effective level (e.g., therapeutic levels have been sustained in the subject for a time period required to produce therapeutic effects in the subject due the single treatment of the first and second compositions). In additional embodiments, the methods further comprise: d) generating a written and/or electronic report that indicates the therapeutic protein and/or biologically active nucleic acid molecule is/are being expressed in the subject at the therapeutic or effective level (e.g., for a certain amount of time). In other embodiments, the report is sent to the treating clinician or practitioner and/or patient from a lab that conducted the test.

In some embodiments, the therapeutic protein and/or biologically active nucleic acid molecule is/are expressed at a level of at least 50 pg/ml . . . 100 . . . 500 . . . 1000 . . . 1500 . . . 4000 . . . 8000 . . . 9500 . . . 1,000,000 pg/ml (1 ug/ml) . . . 1.5 ug/ml or higher, and wherein a blood, serum, or plasma sample (or other biological sample) from the subject is assayed to determine that the therapeutic or effective level is achieved for at least 5 . . . 7 . . . 10 . . . 25 . . . 45 . . . 63 . . . 150 . . . 300 days, or longer, after the administration of the first and second compositions. In other embodiments, the therapeutic protein(s) is/are expressed at a level that is at least 50 pg/ml or at least 100 pg/ml or at least 500, 1,000,000 pg/ml (1 ug/ml) . . . 1.5 ug/ml or higher, and wherein the therapeutic protein is expressed at the level in the subject for at least 5 . . . 7 . . . 10 . . . 25 . . . 45 . . . 63 . . . 150 . . . 300 . . . 350 consecutive days. In certain embodiments, the therapeutic protein and/or biologically active nucleic acid molecule is expressed (e.g., at therapeutic levels) in the subject without clinically significant elevated toxicity (e.g., as measured by ALT (alanine aminotransferase) and/or AST (aspartate aminotransferase)) after at least 48 hours following the administration of the first and second compositions.

In certain embodiments, the therapeutic protein is human G-CSF (e.g., as encoded by SEQ ID NO:1, or sequence with at least 98% identity with SEQ ID NO:1) and is expressed in the subject at a therapeutic level of at least 100 pg/ml as measured in a blood, serum, or plasma sample, wherein the therapeutic protein is expressed in the subject for at least seven days, and wherein the disease, condition, or physiological trait is selected from the group consisting of: neutropenia caused by chemotherapy, non-elevated levels of hematopoietic stem cells in blood of a stem cell donor or recipient, heart degeneration, cerebral ischemia, amyotrophic lateral sclerosis, neutrophil deficiency diseases, and radiation exposure. In particular embodiments, the G-CSF is expressed for at least 5, or 6, or 7 days, but no more than about 10 days (e.g., using drugs, promoter/enhancer combinations, additional expression cassette within the nucleic acid vector or additional expressed proteins to limit production to about 10 days to avoid any toxic neutrophilia-related side effects by expression beyond about 10 days). In other embodiments, the therapeutic protein is Rituximab or similar anti-CD20 antibody or antibody fragment. In some embodiments, the therapeutic protein is human Factor IX or similar protein.

In particular embodiments, the therapeutic protein or proteins and/or biologically active nucleic acid molecule or molecules is/are expressed in the subject for a sufficient amount of time at the therapeutic level to reduce or eliminate the at least one symptom (or all symptoms) without the subject having to receive any other treatment that provides the therapeutic protein(s) and/or biologically active nucleic acid molecule(s) to the subject. In further embodiments, during the sufficient time, the subject does not receive any other specific treatment (e.g., no other specific therapeutic treatment that provides the therapeutic protein or biologically active nucleic acid molecule(s) to the subject). In certain embodiments, the subject has multiple symptoms of a disease or diseases, and wherein the sufficient amount of time is such that all or substantially all of the multiple symptoms of the disease(s) and/or the condition(s) are reduced or eliminated in the subject (e.g., permanently, or for at least 20 days . . . 50 days . . . 200 days . . . 1 year or longer). In other embodiments, during the sufficient time, the subject does not receive the any other disease-specific treatment.

In some embodiments, the first amount of the polycationic structures (e.g., empty cationic liposomes, empty cationic micelles, or empty cationic emulsions) is about 0.01-70, 30-50, or 20-60, μmoles per 1 kilogram of the subject (e.g., 0.01 . . . 1 . . . 10 . . . 20 . . . 40 . . . or 60 μmoles per kilogram). In other embodiments, the ratio of the first amount of the polycationic structures (e.g., empty cationic lipids) to the therapeutically effective amount of the expression vectors is 0.5:1 to 25:1, nmoles of polycationic structures (e.g., empty cationic lipids) to 1 ug of expression vectors (e.g., 0.5:1 . . . 1:1 . . . 4:1 . . . 8:1 . . . 12:1 . . . 17:1 . . . 21:1 . . . or 25:1). In certain embodiments, the ratio of the first amount of the polycationic structures (e.g., empty cationic lipids) to the therapeutically effective amount of the expression vectors is 7:1 to 13:1, nmoles of polycationic structures (e.g., empty cationic lipids) to 1 ug of expression vectors. In particular embodiments, the therapeutically effective amount of the expression vectors is 0.001-8.0 milligrams of the expression vectors per 1 kilogram of the subject (e.g., 0.001 . . . 0.1 . . . 3.0 . . . 4.5 . . . 5.7 . . . 7.1 . . . 8.0 milligrams per kilogram). In some embodiments, the therapeutically effective amount of expression vectors is 0.001 to 1 ug per 1 kilogram of the subject (e.g., 0.001 . . . 0.01 . . . 0.1 . . . 1 ug per kilogram of subject). In certain embodiments, the therapeutically effective amount of the expression vectors is about 0.01-4.0 milligrams of the expression vectors per 1 kilogram of the subject.

In some embodiments, the first nucleic acid sequence encodes the first or first and second, or first, second, and third, therapeutic protein(s). In additional embodiments, the first nucleic acid sequence encodes the biologically active nucleic acid molecule(s). In other embodiments, the subject is a human. In additional embodiments, the expression vectors are CpG-free. In other embodiments, the expression vectors are CpG-reduced. In other embodiments, the therapeutic protein(s) is/are human protein(s) or animal protein(s).

In some embodiments, the polycationic structures do not contain cholesterol (e.g., cholesterol free empty cationic micelles or liposomes). In certain embodiments, the cationic liposomes each comprise at least 60% DOTAP and/or DPTAP (e.g., 60% . . . 75% . . . 85% 95% . . . 98% . . . 100% DOTAP and/or DPTAP). In other embodiments, all or substantially all of the cationic liposomes are multi-lamellar vesicles. In further embodiments, all or substantially all of the cationic liposomes are uni-lamellar vesicles. In further embodiments, the cationic liposomes each comprise at least 99% DOTAP or 99% DPTAP. In further embodiments, the empty cationic liposomes each comprise DOTAP and cholesterol. In additional embodiments, the cationic liposomes each comprise about one-third cholesterol and about two-thirds DOTAP and/or DPTAP. In further embodiments, the first nucleic acid sequence encodes human G-CSF (e.g., as shown in SEQ ID NO:1).

In certain embodiments, the biologically active nucleic acid molecule(s) comprises sequence(s) selected from: shRNA sequence(s), miRNA sequence(s), antisense sequence(s), ribozyme(s), and/or CRISPR single guide RNA sequence(s) (sgRNA). In other embodiments, the CRISPR sgRNA comprises: i) a Cas9 nuclease-recruiting sequence (tracRNA), and ii) a target-specific sequence (crRNA) that hybridizes to a sgRNA target site. In particular embodiments, the biologically active nucleic acid molecule targets human p65 (aka, NF-kappa-B p65 or RELA).

In further embodiments, each of the expression vectors further comprises a second nucleic acid sequence encoding: i) a second therapeutic protein, and/or ii) a second biologically active nucleic acid molecule. In some embodiments, each of the expression vectors further comprises a third nucleic acid sequence encoding: i) a third, and/or fourth therapeutic protein, and/or ii) a third, and/or fourth biologically active nucleic acid molecule. In further embodiments, each of the expression vectors further comprise a first promoter associated with the first nucleic acid sequence, and a second promoter associated with the second nucleic acid sequence, and wherein the first and second promoters are the same or different. In other embodiments, the therapeutic or effective expression level of the first nucleic acid sequence and/or the length of time of the therapeutic or effective expression level, is reduced compared to the expression level or the length of time, when the second nucleic acid is not present and/or expressed from the expression vectors. In other embodiments, the first nucleic acid sequence is expressed at the therapeutic level for at least 5 days, but less than 21 days (e.g., 5 . . . 7 . . . 13 . . . 16 . . . 20 . . . and 21 days). In certain embodiments, the first nucleic acid sequence encodes the therapeutic protein, and wherein the therapeutic protein comprises human G-CSF.

In other embodiments, the expression vector provides the expression at the therapeutic or effective level for a first length of time and/or at a first level of expression when each of the expression vectors comprises a first promoter and first enhancer associated with the first nucleic acid sequence, and wherein the first length of time and/or expression level is altered when a second promoter, different from the first promoter, replaces the first promoter, and/or a second enhancer, different from the second enhancer, replaces the second promoter, on the expression vectors. In other embodiments, the expression at the therapeutic or effective level for a first length of time is for at least 10 . . . 15 . . . 45 . . . 100 . . . 200 . . . 300 days, and wherein replacement with the second promoter and/or second enhancer reduces expression at the therapeutic or effective level to a second length of time that is less than 10 . . . 15 . . . 45 . . . 100 . . . 200 days. In other embodiments, each of the expression vectors comprises a first promoter and a first enhancer, and wherein the first promoter and the first enhancer cause expression at the therapeutic level for at least 5 days, but less than 21 . . . 15 . . . or 10 days. In particular embodiments, the first nucleic acid sequence encodes the therapeutic protein, and wherein the therapeutic protein comprises human G-CSF.

In some embodiments, the methods further comprise administering a drug agent or agents, either in the first and/or second composition, or present in a third composition, wherein the drug agent or agents increase or decrease the expression of the first nucleic acid (e.g., at the therapeutic or effective level, and/or the length of time of the expression at the therapeutic or effective level), compared to when the drug agent or agents are not administered to the subject. In particular embodiments, the drug agent increases the expression level of the first nucleic acid in the subject, and wherein the drug is selected from colchicine, an immunosuppressant, dexamethasone, dexamethasone palmitate, sildenafil, or L-arginine+sildenafil. In certain embodiments, the drug (e.g., dexamethasone or dexamethasone palmitate) is present at between 2.0% and 6.0% of a polycationic structure (e.g., empty cationic lipid composition), such as at 2.0% . . . 2.5% . . . 3.5% . . . 4.5% or 6.0%. In other embodiments, the drug (e.g., dexamethasone or dexamethasone palmitate), is administered to the subject before or after the polycationic structure and vector compositions are administered. In certain embodiments, the polycationic structures (e.g., empty liposomes) present in the first composition have a z-average diameter of about 20-85 nm (e.g., 20 . . . 25 . . . 30 . . . 40 . . . 45 . . . 50 . . . 55 . . . 60 . . . 65 . . . 70 . . . 75 . . . 80 . . . 85 nm). In certain embodiments, the polycationic structures are empty liposomes with a z-average diameter of about 72-76 nm, and are small uni-lammellar vesicles.

In other embodiments, the therapeutic protein is expressed at a level of at least two times higher (or at least 3 or 4 or 5 times higher) when the drug agent is administered to the subject compared to when the drug agent is not administered to the subject. In particular embodiments, the drug agent decreases the expression level of the first nucleic acid sequence, and wherein the drug agent is L-arginine. In further embodiments, the therapeutic protein is expressed at a level of at least two times (or at least three times or four times) lower when the drug agent is administered to the subject compared to when the drug agent is not administered to the subject. In some embodiments, the drug agent comprises an anti-inflammatory agent. In additional embodiments, the drug agent is selected from the group consisting of: amlexanox, chloroquine, valproic acid, theophylline, DHA, prostaglandin, and SAHA.

In further embodiments, the expression vectors are free of operable matrix attachment region (MAR) sequences. In certain embodiments, the expression vectors are free of operable EBNA-1 and/or EBV viral sequences. In certain embodiments the subject's blood pressure, immediately prior to said administering said first and second compositions, is not altered (e.g., no physical transfection aids are applied to the subject to attempt to increase expression of the first nucleic acid sequence).

In particular embodiments, the therapeutic level and/or effective level is at least 150 . . . 100 . . . 500 . . . 1000 . . . 1500 . . . 5000 . . . 1,000,000 pg/ml (1 ug/ml) . . . 1.5 ug/ml or higher, and wherein a blood, serum, or plasma sample (or other biological sample) from the subject is determined to be at the therapeutic level and/or effective level at least 7 . . . 10 . . . 25 . . . 45 . . . 63 . . . 150 . . . 300 . . . 400 days or more after the administration of the first and second compositions. In particular embodiments, the sample from the subject is tested with an ELISA assay or by mass spectrometry to determine the expression level.

In some embodiments, the methods further comprise administering a therapeutically effective amount of neutral liposomes to the subject, wherein the neutral liposomes are present in the first and/or second composition, and/are administered in a third composition, and wherein the therapeutically effective amount of neutral liposomes are administered to the subject prior to the administering the second composition. In certain embodiments, the neutral liposomes comprise at least material selected from: phospholipon 90H, hydrogenated soy PC, stearic and palmitic. In other embodiments, the therapeutically effective amount of neutral liposomes are present in the first composition or present in a third composition administered to the subject. In further embodiments, the neutral liposomes are multilamellar vesicles or extruded to 0.2 or 0.1 um. In particular embodiments, administering the therapeutically effective amount of the neutral liposomes causes expression of the first therapeutic protein and/or the biologically active nucleic acid molecule in the subject that is at least 3 . . . 4 . . . 25 . . . 100 . . . 350 . . . or 600 times higher than occurs when the neutral liposomes are not administered to the subject. In certain embodiments, the ratio of empty cationic liposomes to the neutral liposomes administered to the subject is between about 2:1 and 1:5 (e.g., 2:1 . . . 1:1 . . . 2:5 . . . 1:5).

In some embodiments, provided herein are methods of expressing a first therapeutic protein and/or a biologically active nucleic acid molecule in a subject comprising: a) administering a first composition to a subject, wherein the first composition comprises an anti-inflammatory agent; and b) administering or initiating administration of, a second composition to the subject within about 2 minutes . . . 20 minutes . . . 1 hour . . . 24 hours . . . 5 days . . . 7 days . . . 9 days or more of administering the first composition, wherein the second composition comprises a therapeutically effective amount of polyplexes, wherein each polyplex comprises an expression vector and polyethylenimine, wherein the expression vector is CpG-free or CpG-reduced, wherein each expression vector comprises a first nucleic acid sequence encoding: i) a first therapeutic protein (and/or first and second proteins), and/or ii) a first (and/or first and second) biologically active nucleic acid molecule, and wherein, as a result of administering the first composition and administering the second composition, the first therapeutic protein and/or the biologically active nucleic acid molecule is/are expressed in the subject. In further embodiments, the subject has at least one symptom of a disease or condition, or has at least one physiological trait desired to be altered, and wherein the first therapeutic protein and/or the biologically active nucleic acid molecule is expressed at a therapeutic level with respect to the disease, condition, or physiological trait to be altered. In some embodiments, the anti-inflammatory agent is selected from the group consisting of amlexanox, chloroquine, and suberanilohydroxamic acid (SAHA).

In some embodiments, the expression vector comprises a plasmid or other non-viral vector. In further certain embodiments, the administration in step b) is accomplished by systemically administering the second composition.

In some embodiments, provided herein are systems or kits comprising: a) a first composition comprising a first amount of polycationic structures (e.g., empty cationic liposomes, empty cationic micelles, or empty cationic emulsions), wherein the first composition is free, or essentially free, of nucleic acid molecules; and b) a second composition comprises a therapeutically effective amount of expression vectors (e.g., non-viral and not associated with liposomes or other carrier molecules), wherein the expression vectors are CpG-free or CpG-reduced, wherein each of the expression vectors comprises a first nucleic acid sequence encoding: i) a first therapeutic protein or non-therapeutic protein, and/or ii) a first biologically active nucleic acid molecule. In other embodiments, the expression vectors are a naked, non-viral expression vectors (e.g., plasmid). In certain embodiments, at least one of the following applies: i) wherein the ratio of the first amount of the polycationic structure (e.g., empty cationic liposome) to the therapeutically effective amount of expression vectors is 2:1 to 25:1 or 5:1 to 25:1; ii) wherein 2.0% to 6.0% of the first composition comprises dexamethasone palmitate; iii) wherein the first composition further comprises neutral lipid, and iv) wherein the polycationic structures comprise empty liposomes, and wherein the empty liposomes present in the first composition have a z-average diameter of about 20-85 nm (e.g., 20 . . . 25 . . . 30 . . . 40 . . . 45 . . . 50 . . . 55 . . . 60 . . . 65 . . . 70 . . . 75 . . . 80 . . . 85 nm). In certain embodiments, the vectors are viral vectors (e.g., AAV or adeno viral vectors). In particular embodiments, the therapeutic protein is human G-CSF (e.g., as shown in SEQ ID NO:1).

In particular embodiments, the first amount of the polycationic structure (e.g., empty cationic liposomes) is between 0.1 to 7.0 millimoles (e.g., 0.1 . . . 5.0 . . . 7.0 millimoles) or 1.5 and 5.0 millimoles (e.g., suitable amount for administration to a human subject). In other embodiments, the ratio of the first amount of the polycationic structure (e.g., empty cationic liposome) to the therapeutically effective amount of the expression vectors is 0.5:1 to 25:1, nmoles of empty cationic lipid to 1 ug of expression vectors (e.g., 0.5:1 . . . 1:1 . . . 5:1 . . . 10:1 . . . 15:1 . . . 25:1). In some embodiments, the ratio of the first amount of the polycationic structure (e.g., empty cationic lipid) to the therapeutically effective amount of the expression vectors is 7:1 to 13:1, nmoles of polycationic structure to 1 ug of expression vectors (e.g., 7:1 . . . 10:1 . . . or 13:1). In other embodiments, the therapeutically effective amount of the expression vectors is between 0.1 and 800 milligrams (e.g., suitable amount for administration to a human subject, such as when the vector is a plasmid). In certain embodiments, the amount is 1 . . . 25 . . . 400 . . . or 800 milligrams of expression vectors for human administration.

In other embodiments, the first nucleic acid sequence encodes the first therapeutic protein. In additional embodiments, the first nucleic acid sequence encodes the biologically active nucleic acid molecule. In particular embodiments, the expression vectors are CpG-free. In other embodiments, the expression vectors are CpG-reduced. In further embodiments, the first therapeutic protein is a human protein. In other embodiments, the first nucleic acid sequence encodes the therapeutic protein, and wherein the therapeutic protein comprises human G-CSF, Rituximab, a monoclonal antibody or monoclonal antibody fragment (e.g., Fab), or human Factor IX.

In certain embodiments, the empty cationic liposomes, micelles, or emulsions, each comprise at least 60% DOTAP and/or DPTAP (e.g., 60% . . . 75% . . . 85% . . . 95% . . . 98% 100% DOTAP and/or DPTAP), and may be cholesterol-free (e.g., no detectable cholesterol in the composition). In other embodiments, all or substantially all of the empty cationic liposomes, micelles, or emulsions are multilamellar vesicles. In further embodiments, all or substantially all of the empty cationic liposomes, micelles, or emulsions are either unilamellar, multilamellar, or oligolamellar vesicles. In further embodiments, the empty cationic liposomes, micelle, or emulsions each comprise at least 99% DOTAP or at least 99% DPTAP, and may be cholesterol free. In further embodiments, the empty cationic liposomes each comprise DOTAP and/or DPTAP and cholesterol. In additional embodiments, the empty cationic liposomes, micelles, or emulsions each comprise about one-third cholesterol and about two-thirds DOTAP and/or DPTAP.

In certain embodiments, the first biologically active nucleic acid molecule comprises a sequence selected from: an siRNA or shRNA sequence, a miRNA sequence, an antisense sequence, and a CRISPR single guide RNA sequence (sgRNA). In other embodiments, the CRISPR sgRNA comprises: i) a Cas9 nuclease-recruiting sequence (tracRNA), and ii) a target-specific sequence (crRNA) that hybridizes to a sgRNA target site.

In further embodiments, each of the expression vectors further comprises a second nucleic acid sequence encoding: i) a second therapeutic protein, and/or ii) a second biologically active nucleic acid molecule. In further embodiments, each of the expression vectors further comprise a first promoter associated with the first nucleic acid sequence, and a second promoter associated with the second nucleic acid sequence, and wherein the first and second promoters are the same or different.

In some embodiments, the kits and systems further comprise a first container and a second container, and wherein the first composition is present in the first container and the second composition is present in the second container. In other embodiments, kits and systems further comprise a packaging component (e.g., cardboard box, plastic pouch, etc.), wherein the first container and the second container are inside the packaging component.

In certain embodiments, the kits and systems further comprise a drug agent or drug agents, wherein the drug agent(s) are present in the first and/or second compositions, or is present in a third composition. In additional embodiments, the drug agent is selected from colchicine, an immunosuppressant, dexamethasone, sildenafil, L-arginine, or L-arginine+sildenafil. In further embodiments, the drug agent comprises an anti-inflammatory agent. In further embodiments, the drug agent is selected from the group consisting of: amlexanox, valproic acid, theophylline, chloroquine, and SAHA.

In particular embodiments, the expression vectors are free of operable matrix attachment region (MAR) sequences. In additional embodiments, the expression vectors are free of operable EBNA-1 and/or EBV viral sequences.

In certain embodiments, the kits and systems further comprise a therapeutically effective amount of neutral liposomes, wherein the neutral liposomes are present in the first and/or second compositions, or is present in a third composition. In additional embodiments, the therapeutically effective amount of neutral liposomes are present in the first composition. In other embodiments, the neutral liposomes are multilamellar or oligo- or uni-lamellar vesicles. In further embodiments, the ratio of empty cationic liposomes or micelles to the neutral liposomes is between about 2:1 and 1:5 (e.g., 2:1 . . . 1:1 . . . 3:5 . . . 1:5).

In some embodiments, provided herein are a first composition and a second, separate, composition for combined use in the treatment of a disease amenable to treatment with in vivo expression of a first therapeutic protein and/or biologically active nucleic acid molecule, wherein the first composition comprises a first amount of polycationic structures (e.g., empty cationic liposomes, empty cationic micelles, or empty cationic emulsions), wherein the first composition is free, or essentially free, of nucleic acid molecules; and b) a second composition that comprises a therapeutically effective amount of expression vectors, wherein the expression vectors are CpG-free or CpG-reduced, wherein each of the expression vectors comprises a first nucleic acid sequence encoding: i) a first therapeutic protein, and/or ii) a first biologically active nucleic acid molecule.

In certain embodiments, provided herein are methods of expressing a first therapeutic protein and/or a biologically active nucleic acid molecule in a subject comprising: a) administering a first composition to a subject, wherein the first composition comprises a first amount of polycationic structures (e.g., empty cationic liposomes, empty cationic micelles, or empty cationic emulsions), and wherein the first composition is free, or essentially free, of nucleic acid molecules; and b) administering a second composition to the subject within about 100 minutes or 200 minutes of administering the first composition, wherein the second composition comprises a therapeutically effective amount of non-viral expression vectors, wherein the expression vectors are CpG-free or CpG-reduced, wherein the expression vectors each comprise a first nucleic acid sequence encoding: i) a first therapeutic protein, and/or ii) a first biologically active nucleic acid molecule, and wherein, as a result of administering the first composition and administering about the second composition, the first therapeutic protein and/or the biologically active nucleic acid molecule is/are expressed in the subject at a level above (e.g., at least 150 . . . 300 . . . 575 . . . 1000 . . . 1500 . . . 2000 . . . 5000 . . . or 1,000,000 pg/ml) (e.g., as measured in a serum sample from the subject (e.g., after 7 . . . 25 . . . 50 days from the first and second administrations).

In certain embodiments, provided herein are methods comprising: administering a composition to a subject comprising a therapeutically effective amount of non-viral expression vectors that are CpG-free or CpG-reduced and comprise a first nucleic acid sequence encoding: i) a first therapeutic protein, and/or ii) a first biologically active nucleic acid molecule, and wherein, as a result of administering the first and second compositions, the first therapeutic protein and/or the biologically active nucleic acid molecule is/are expressed in the subject at a level above 100 pg/ml (e.g., at least 150 . . . 400 . . . 1200 . . . 2000 . . . 5000 . . . or more than 1,000,000 pg/ml) (e.g., as measured in a serum sample from the subject (e.g., after 7 . . . 25 . . . 50 days from the first and second administrations).

In certain embodiments, the polycationic structures comprises empty cationic liposomes, micelles, or emulsions. In other embodiments, the polycationic structures comprises one or more of the following, either alone or combined with polycationic structures: linear or branched polyethyleneimine, dendrimers (e.g., 4th generation pamaam dendrimer based on ethylene diamine, polylysine, polyarginine, and protamine sulfate), poly-lysine, and protamine sulfate. In certain embodiments, the polycationic structures are provided as a cationic emulsion. In particular embodiments, the surfactants in the emulsions are selected from: cetylpyridinium chloride, cetyltrimethylammonium bromide or the like. In other embodiments, the emulsions further comprise a neutral component, such as tweens, spans and triglycerides. In particular embodiments, the emulsions comprises a cationic lipid, such as, for example, DOTAP, DPTAP, DOTMA, or DDAB. In some embodiments, the emulsions are self-emulsifying emulsions or microemulsions (SEDDS, SMEDDS).

In some embodiments, provided herein are methods of expressing a first and second proteins and/or first and second biologically active nucleic acid molecules in a subject comprising: a) administering a first composition to a subject, wherein the subject has at least one symptom of a disease or condition, or has at least physiological trait to be altered, wherein said first composition comprises a first amount of polycationic structures, and wherein said first composition is free, or essentially free, of nucleic acid molecules; and b) administering a second composition to said subject within about 100 minutes of administering the first composition, wherein the second composition comprises a therapeutically effective amount of expression vectors, wherein the expression vectors are non-viral and are CpG-free or CpG-reduced, wherein the expression vectors each comprise: i) a first expression cassette encoding: A) a first protein, and/or B) a first biologically active nucleic acid molecule, and ii) a second expression cassette encoding: A) a second protein and/or B) a second biologically active nucleic acid molecule. In certain embodiments, as a result of the administering the first composition and the administering the second composition, the first and second proteins and/or said first and second biologically active nucleic acid molecule is/are expressed in the subject at a therapeutic level with respect to the disease or condition, or at an effective level sufficient to alter said physiological or disease trait.

In particular embodiments, the first protein comprises a monoclonal antibody light chain, and the second protein comprises a heavy chain of said monoclonal antibody. In other embodiments, the first and second expression cassettes both comprise regulatory elements. In additional embodiments, the regulatory elements are the same or different in said first and second expression cassettes.

### DESCRIPTION OF THE FIGURES

FIG. 1 shows a schematic representation of the various CPG-free plasmid constructs used in Example 1.

FIG. 2 shows the CpG-free modified nucleic sequence of h-GCSF (SEQ ID NO:1) and the amino acid sequence of h-GCSF (SEQ ID NO:2). The positions where CpG di-nucleotides have been eliminated are shown in underline in SEQ ID NO:1.

FIG. 3 shows a graph of serum human G-CSF levels produced in mice by sequential, IV cationic liposome injection followed by IV DNA vector injection.

FIG. 4 shows a histogram of WBC and absolute neutrophil counts, 21 days after sequential, IV cationic liposome then DNA vector injection.

FIG. 5 shows serum human G-CSF levels produced in mice by sequential IV injection of either single or dual cassette, hG-CSF single plasmid vectors.

FIG. 6 shows serum hG-CSF levels in mice, 21 days after IV injection of cationic liposomes, then DNA containing different promoter-enhancer combinations linked to the hG-CSF gene.

FIG. 7 shows mouse lung luciferase levels, 7 days after sequential IV injection of cationic liposomes, then single cassette, EF1-luciferase DNA alone or together with a particular drug.

FIG. 8 shows mouse lung luciferase levels, 7 days after sequential IV injection of cationic liposomes, then dual cassette, EF1-hG-CSF-EF1-luciferase DNA alone or with certain drug(s).

FIG. 9 shows mouse lung luciferase levels, 10 days after sequential IV injection of cationic liposomes, then a DNA vector containing one of a series of different promoter-enhancer combinations, each either with or without MARs, and all linked to the luciferase gene.

FIG. 10 shows mouse lung luciferase levels, 1 or 5 days after IV injection of PEI: EF-1 Luc DNA complexes or sequential IV injection of cationic liposomes, then the identical EF-1 Luc DNA.

FIG. 11 shows mouse lung luciferase levels, 1 day after sequential IV injection of one of seven different cationic liposome formulations, then single cassette, EF1-luciferase DNA.

FIG. 12 shows mouse spleen luciferase levels, 1 day after IV injection of PEI: EF-1 Luc DNA complexes alone or mixed with one of four different drugs.

FIG. 13 shows mouse lung luciferase levels, 1 or 7 days after sequential IV injection of cationic liposomes, then one of a series of dual cassette, EF-1-Luc-hG-CSF DNA vectors.

FIG. 14 shows serum human G-CSF levels produced in mice, 1 day after sequential, IV cationic liposome injection, with or without co-injection of neutral liposomes, followed by IV injection of a dual cassette, single plasmid vector.

FIG. 15 shows serum human G-CSF levels produced in mice, 1 or 7 days after sequential, IV cationic liposome co-injection with neutral liposomes, followed by IV injection of a dual cassette plasmid vector.

FIG. 16 shows serum human G-CSF levels produced in mice 7 days after sequential, IV cationic liposome injection with SUV, 0.1 μm extruded or MLV cationic liposomes, followed by IV injection of a EF1-hG-CSF plasmid vector.

FIG. 17 shows results from Example 2, wherein one sequential IV injection of cationic liposomes followed by a CPG-free, human G-CSF DNA vector produces supra-therapeutic human G-CSF serum protein levels in mice for at least the next 428 days.

FIG. 18 shows results from Example 3, wherein it was shown that one sequential IV injection of cationic liposomes followed by a CPG-free, human G-CSF DNA vector produces supra-therapeutic human G-CSF serum protein, WBC and ANC levels, with normal ALT (alanine aminotransferase) and AST (aspartate aminotransferase) in rats.

FIG. 19 shows results from Example 3, where it was shown that cationic liposomes generated from DPTAP mediate in vivo transfection.

FIG. 20 shows that toxicity as measured by serum levels of alanine aminotransferase (ALT) and aspartate aminotransferase (AST) are elevated 2 to 5 fold at 24 hrs and return to control levels by 48 hours after sequential injection of cationic liposomes then plasmid DNA.

FIG. 21 shows incorporation of 2.5 mole % dexamethsone palmitate (DexP) into cationic DOTAP liposomes increases expression of hG-CSF at 24 hours after sequential IV injection while simultaneously reducing toxicity, as measured by ALT levels to close to background levels.

FIG. 22 shows that IV injection of DOTAP liposomes containing 2.5% dexamethasone palmitate reduces toxicity, as measured by ALT levels to background levels while significantly increasing human G-CSF protein levels.

FIG. 23 shows pre- and post-injection of Dexamethasone significantly increases hG-CSF protein levels while reducing toxicity, as measured by ALT levels to close to background levels.

FIG. 24 shows manipulating Lipid: DNA Ratios increases hG-CSF levels while reducing toxicity, as measured by ALT levels to background levels.

FIG. 25 shows that IP pre-injection of dexamethasone, followed by 2.5 mole % dexamethsone palmitate in cationic DOTAP liposomes then a dual cassette, single plasmid DNA vector encoding Rituximab significantly increases serum Rituximab levels over time in mice.

FIG. 26 shows dexamethasone pre-injection followed by one IV sequential injection of DexP cationic liposomes plus neutral lipid then a dual cassette, single plasmid DNA vector encoding Rituximab produces extended serum levels of fully functional Rituximab protein in mice.

FIG. 27 shows mouse serum tested at 6 weeks following IV sequential injection of a dual cassette, single plasmid Rituximab DNA vector binds target CD20+ human B lymphoma (Raji) cells similarly to recombinant Rituximab protein.

FIG. 28 Rituximab protein in serum from Rituximab DNA vector-injected mice induces lysis of Raji CD20+ human B cells at levels similar to recombinant Rituximab.

FIG. 29 shows IP pre-injection of dexamethasone, then neutral lipid plus 2.5 mole % dexamethsone palmitate in DOTAP liposomes increases serum Rituximab levels over time in rats.

FIG. 30 shows codon-optimization of Rituximab dual cassette, single plasmid DNA vectors further increases serum Rituximab levels 24 hours after sequential IV injection.

FIG. 31 shows that one sequential, IV cationic liposome injection of codon-optimized dual cassette, single plasmid Rituximab DNA vectors produces extended serum Rituximab levels.

FIG. 32 shows that pre-injection of selected drugs significantly increases serum Rituximab levels produced by sequential IV, cationic liposome injection of a codon-optimized dual cassette, single plasmid Rituximab DNA vector.

FIG. 33 shows sequential IV injection of a single cassette DNA vector encoding the Rituximab heavy and light chains separated by a 2A self cleaving peptide sequence produces significant serum Rituximab protein levels.

FIG. 34 shows that manipulating Lipid: DNA Ratios increases serum Rituximab levels while reducing toxicity, as measured by ALT levels to close to background levels.

FIG. 35 shows that pretreatment with either valproic acid or theophylline significantly increases serum human factor nine levels produced by sequential IV, cationic liposome injection of a codon-optimized, EF-1-driven plasmid vector encoding a human factor IX cDNA.

FIG. 36 shows the arrangement of the Rituximab (anti-CD20) dual cassette plasmids used in the Examples. In this figure, the following abbreviations apply: M: Mar (M1: β-Glo, M2: 21q21 and M3: IFNβ); K: Kozak Sequence (K1: AAGCTTTCC, SEQ ID NO:3; K2: AAGCCACC, SEQ ID NO:4); Enhancer: mCMV or hCMV; Promoter: CMV or EF1; 5′UTR: 1126 or htlv; H: Chimeric Heavy Chain cDNA; L: Chimeric Light Chain cDNA; and pA: poly A

FIG. 37 shows the arrangement of the Rituximab (anti-CD20) single (bisicontronic) plasmid used in the Examples. In this figure, the following abbreviations apply: K: Kozak Sequence (K1: AAGCTTTCC K2, SEQ ID NO:3; AAGCCACC, SEQ ID NO:4); Enhancer: mCMV or hCMV; Promoter: CMV or EF1; 5′UTR: 1126 or htlv

H: Chimeric Heavy Chain cDNA; L: Chimeric Light Chain cDNA; F: Furin (F1: RHQR; F2: RAKR); 2A Peptide: P2A or F2A; and pA: polyA.

FIG. 38 shows the arrangement of the human Factor IX plasmids used in the Examples. The following abbreviations apply in this figure: M: Mar (M1: β-Glo, and M3: IFNβ); Kozak2: Kozak Sequence2 (AAGCCACC, SEQ ID NO:4); Enhancer: mCMV; Promoter: EF1; 5′UTR: I126; hFIX: human Factor XI cDNA; and pA: polyA

FIG. 39 shows one example (No. 8, G4) of a bicistronic, single cassette plasmid construct (SEQ ID NO:5) used in the Examples below that expresses the heavy and light chains (underlined) of Rituximab.

FIG. 40 shows one example (No. 2) of a dual cassette non-optimized anti-CD20 CpG free plasmid construct (SEQ ID NO:6) used in the Examples below that expresses the heavy and light chains (underlined) of Rituximab.

FIG. 41 shows one example (No. 4) of a dual cassette non-optimized anti-CD20 CpG free plasmid construct (SEQ ID NO:7) used in the Examples below that expresses the heavy and light chains (underlined) of Rituximab.

FIG. 42 shows one example (No. 4) of a dual cassette MAR-less optimized anti-CD20 plasmid construct (SEQ ID NO:8) used in the Examples below that expresses the heavy and light chains (underlined) of Rituximab.

FIG. 43 shows one example (No. 6) of a dual cassette MAR-containing optimized anti-CD20 plasmid construct (SEQ ID NO:9) used in the Examples below that expresses the heavy and light chains (underlined) of Rituximab.

FIG. 44 shows one example (No. 4) of a plasmid construct (SEQ ID NO:10) used in the Examples below that expresses human Factor IX.

### DEFINITIONS

As used herein, the phrase “CpG-reduced” refers to a nucleic acid sequence or expression vector that has less CpG di-nucleotides than present in the wild-type versions of the sequence or vector. “CpG-free” means the subject nucleic acid sequence or vector does not have any CpG di-nucleotides. An initial sequence, that contains CpG dinucleotides (e.g., wild-type version of human G-CSF), may be modified to remove CpG dinucleotides by altering the nucleic acid sequence. Such CpG di-nucleotides can be suitably reduced or eliminated not just in a coding sequence, but also in the non-coding sequences, including, e.g., 5′ and 3′ untranslated regions (UTRs), promoter, enhancer, polyA, ITRs, introns, and any other sequences present in the nucleic acid molecule or vector.

As used herein, “empty liposomes” refers to liposomes that do not contain nucleic acid molecules but that may contain other bioactive molecules (e.g., liposomes that are only composed of the lipid molecules themselves, or only lipid molecules and a small molecule drug).

As used herein, “empty cationic micelles” refers to cationic micelles that do not contain nucleic acid molecules but that may contain other bioactive molecules (e.g., micelles that are only composed of lipid and surfactant molecules themselves, or only lipid and surfactant molecules and a small molecule drug).

As used herein, “empty cationic emulsions” refers to cationic emulsions or micro-emulsions that do not contain nucleic acid molecules but that may contain other bioactive molecules.

### DETAILED DESCRIPTION

The present invention provides compositions, systems, kits, and methods for generating expression of a protein or biologically active nucleic acid molecule in a subject (e.g., at therapeutic levels for extended periods of time). In certain embodiments, systems and kits are provided that comprise a first composition comprising a first amount polycationic structures (e.g., empty cationic liposomes, empty cationic micelles, or empty cationic emulsions), and a second composition comprising a therapeutically effective amount of expression vectors (e.g., non-viral expression vectors not associated with liposomes) that are CpG-free or CpG-reduced, where the expression vectors comprise a first nucleic acid sequence encoding: i) a first therapeutic protein, and/or ii) a first biologically active nucleic acid molecule. In other embodiments, such first and second compositions are sequentially administered (e.g., systemically) to a subject such that the first therapeutic protein and/or the biologically active nucleic acid molecule is/are expressed in the subject (e.g., at a therapeutic level, for at least 5 or at least 50 days, such that a disease or condition is treated or a physiological trait is altered).

Work conducted during the development of embodiments of the present disclosure has shown that a single injection (e.g., intravenous injection) of cationic liposomes, followed shortly thereafter by injection (e.g., intravenous injection) of CpG-free vectors encoding a therapeutic protein produces circulating protein levels many times (e.g., 10-20 times higher) than the therapeutic serum level for the protein for a prolonged period. Such administration also increased circulating neutrophil counts many fold weeks after the treatment.

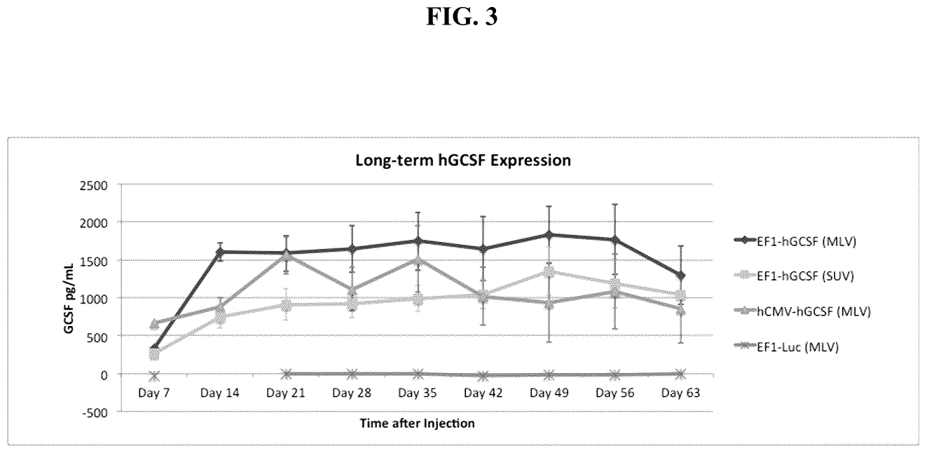

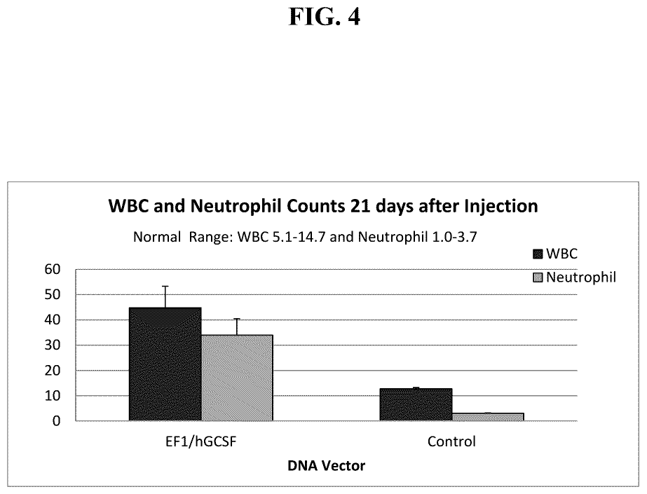

Work conducted during the development of embodiments of the present disclosure (e.g., as shown in Example 1 below) has shown that a single intravenous injection of cationic liposomes, followed two minutes later by intravenous injection of CpG-free plasmid vectors encoding human granulocyte-colony stimulating factor (hG-CSF) produces circulating hG-CSF protein levels 10-20 times higher than the therapeutic serum hG-CSF level (greater than or equal to 100 pg/ml) for at least 63 days (see, FIG. 3). Such administration also increased circulating neutrophil counts 10 fold, 3 weeks following intravenous injection into mice (FIG. 4). In contrast, one systemic injection of cationic liposome-DNA complexes containing a similar, but CpG-containing) hG-CSF plasmid vector was unable to produce detectable (>20 pg/ml) hG-CSF protein levels even at day 3 after injection, and failed to increase neutrophil counts at any point after injection (see, Tu et al., JBC, 275 (39): 30408-30416, 2000; herein incorporated by reference in its entirety). Moreover, the approach presented in Example 1 that was used to prolong expression at therapeutic levels of human G-CSF did not appear to cause significant toxicity in the mice.

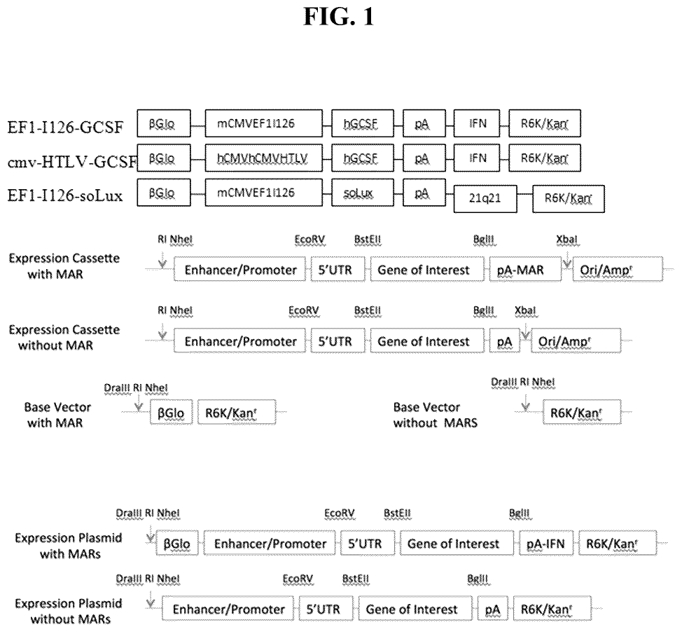

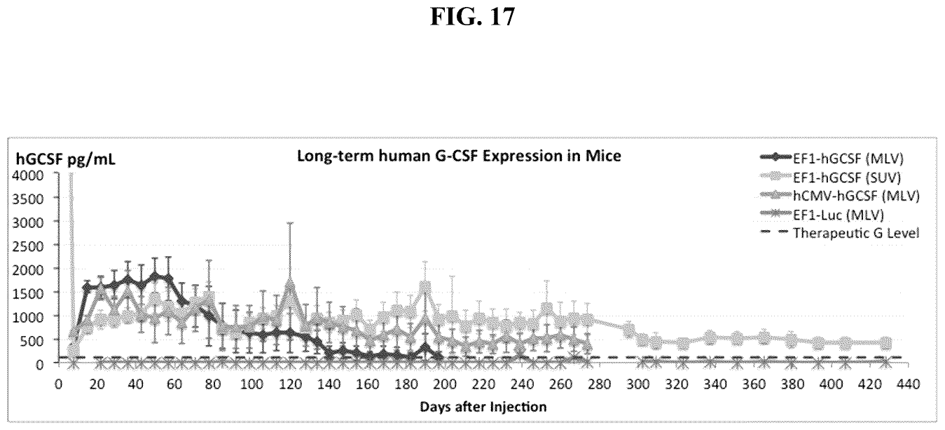

Thus, the approach provided herein for expression in vivo overcomes the critical limitation that has up to now precluded the successful therapeutic application of systemic non-viral gene delivery. Namely, its inability to express delivered genes at therapeutic levels for the extended periods generally required to produce important therapeutic or physiological endpoints. As shown in Example 1, embodiments of the methods provided herein accomplish such long lasting expression of a therapeutic protein with non-viral vectors without having to incorporate viral genes into the vectors. This is important as other approaches relied on the insertion of at least one viral gene plus the viral DNA sequence to which its protein product binds (the EBNA-1 gene together with the EBV family of repeat sequences inserted into the DNA vector) has been required in order to overcome this transient gene expression limitation (see, Tu et al., above). Moreover, in addition to the high hG-CSF protein levels found after 63 days in Example 1 (FIG. 1), similar high levels of expression were measured and found on days 14, 21, 28, and 49 after injection, indicating that once achieved, these high therapeutic levels are maintained longer term. Also, Example 2 shows, in FIG. 17, over 400 days of high levels of expression. This high level and long term expression is significantly better than the mRNA approach provided by MODERNA, which, as shown in FIG. 3 of U.S. Pat. No. 8,754,062 for hG-CSF, only produced therapeutic levels of up to 4 days after a single IV injection.

In addition, the systems, methods, and compositions provided herein provide a versatile (e.g., non-viral) gene delivery and expression platform that can much more precisely control the duration of expression of delivered genes at therapeutic levels. This ability to control the duration of expression of delivered genes addresses another up to now critical unmet need within the gene therapy field, the ability to control the duration at which proteins are expressed at therapeutic levels. Specifically, there is now a wide and expanding spectrum of FDA-approved, recombinant, secreted human protein therapies. Different approved protein therapies must be present at therapeutic levels for very different durations in order to both effectively and safely treat patients. Recommended treatment durations of different protein therapies vary from less than two weeks (hG-CSF) to the lifetime of the patient (factor IX). For example, recombinant human G-CSF protein, Neupogen, is given daily for only the first 10 days of each three-week chemotherapy cycle. Serum hG-CSF levels return to baseline approximately 14 hours after each daily Neupogen dose. This 10 day treatment schedule is used because its neutrophil increasing effect is indicated only during this approximately 10 day period of chemotherapy-induced neutropenia. G-CSF elevation from days 11 to 21 is generally not beneficial, as the patient's own neutrophil producing capacity returns. Giving Neupogen beyond day 10 can cause toxic, neutrophilia-related side effects. In contrast, anti-TNF antibodies are routinely administered for months or years, and factor IX replacement for the lifetime of the patient. Thus, different proteins must be produced at therapeutic levels for different durations, from less than two weeks to the lifetime of the patient. Therefore, a gene therapy approach that can control the duration of gene expression at therapeutic levels it produces in patients achieves therapeutic endpoints while avoiding toxic side effects for a wide spectrum of now FDA-approved, human therapeutic proteins. Provided herein are various technologies that can be employed to provide this control. Five exemplary approaches are described below.

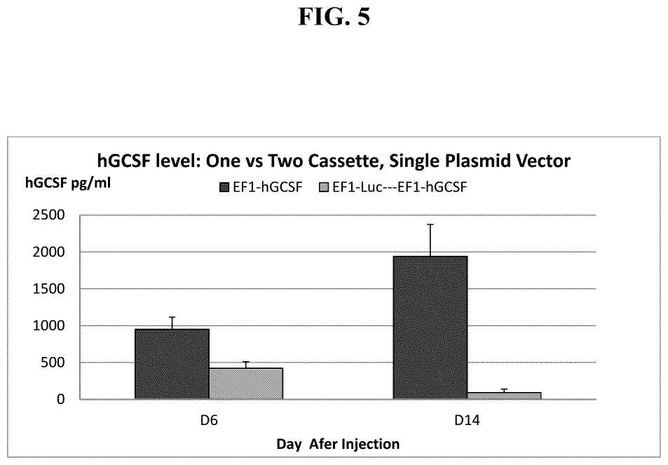

First, in certain embodiments, a second expression cassette is inserted into a single plasmid DNA vector or other vector. As shown in Example 1, in contrast to the single expression cassette hG-CSF plasmid vector that was used, which produces therapeutic hG-CSF levels for at least 63 days (FIG. 3), adding the second cassette limited therapeutic levels of hG-CSF protein produced to less than two weeks in mice (FIG. 5). Of note, the second expression cassette which drives the luciferase gene, is also expressed at high, controllable levels in IV injected mice.

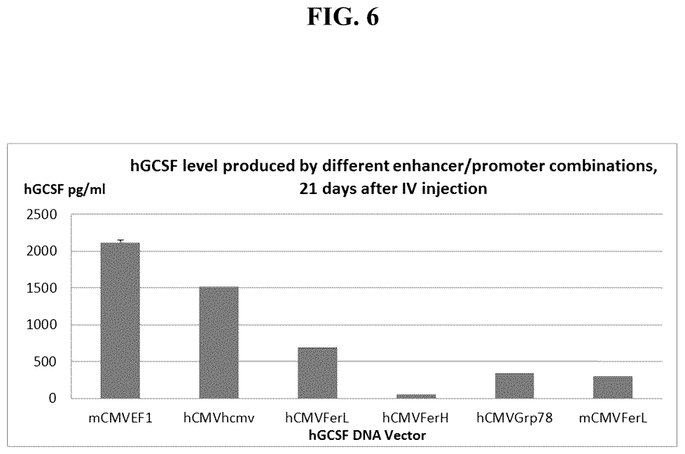

Second, as shown in Example 1, a series of different, CPG-free promoter-enhancer combinations were generated in single cassette plasmid vectors that express hG-CSF at therapeutic levels for a range of different durations following a single IV injection in mice (FIG. 6). Of note, multi-expression cassette, single plasmid DNA vectors that contain different cassettes incorporating different promoter enhancer combinations are capable of expressing different therapeutic proteins at different levels for different durations from a single DNA vector. This allows a single DNA vector to express multiple different therapeutic proteins (e.g., one, two, three, four, five, six or more therapeutic proteins). Each individual protein is then expressed for the required duration at its appropriate therapeutic level. Such an approach is one way to overcome the prohibitive costs now incurred by combining two or more recombinant protein therapies in a single patient.

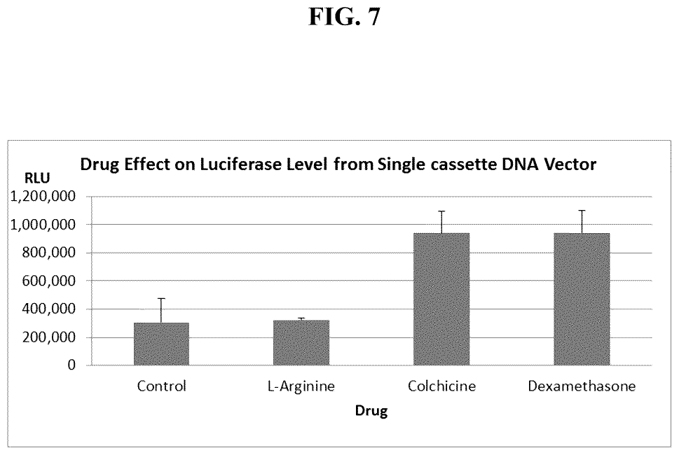

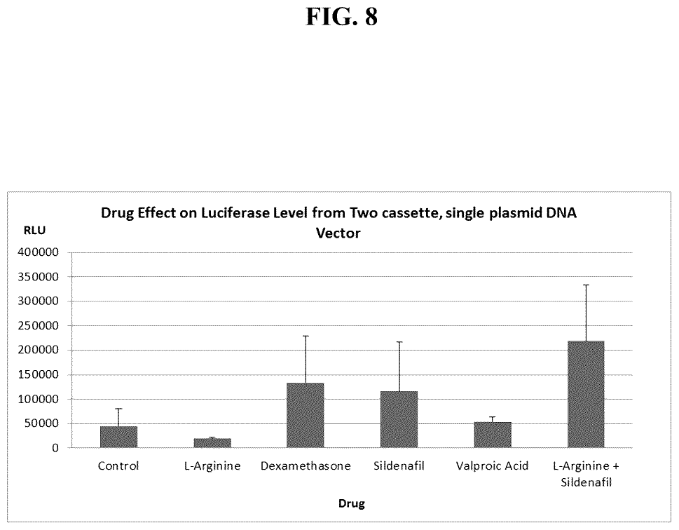

Third, as described in Example 1, it was shown that co-injecting now FDA-approved drugs, singly or in selected combinations with the cationic liposomes can selectively either increase or decrease the level/duration of expression of the gene subsequently delivered by sequential IV injection in mice (FIGS. 7 and 8).

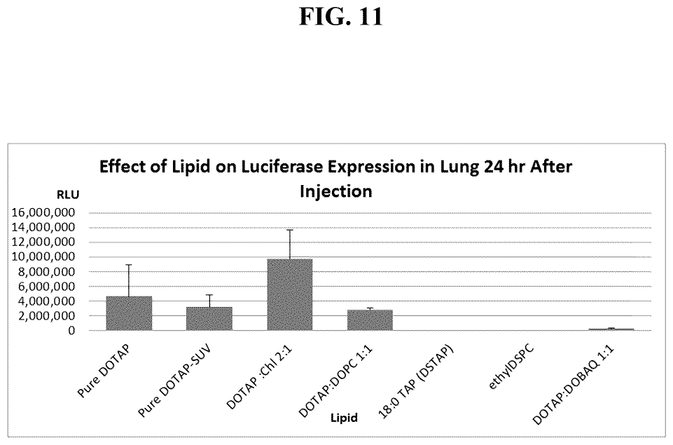

Fourth, as demonstrated in Example 1, varying the cationic liposome size, as well as the lipid composition can also control the level and duration of expression of genes delivered by sequential cationic liposome then DNA injection (FIG. 11).

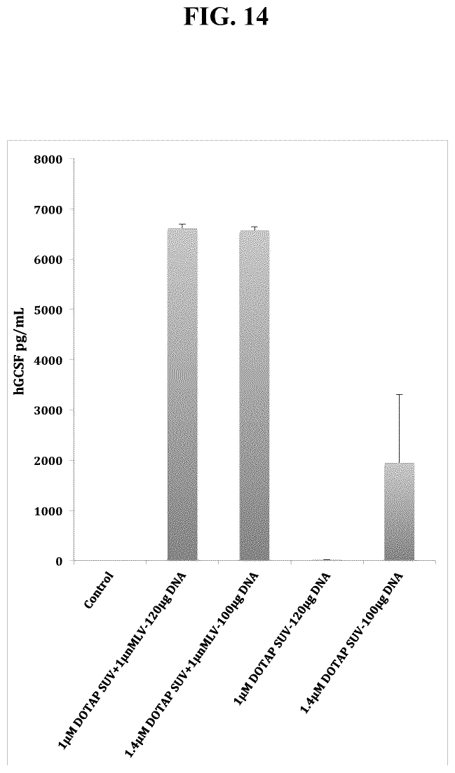

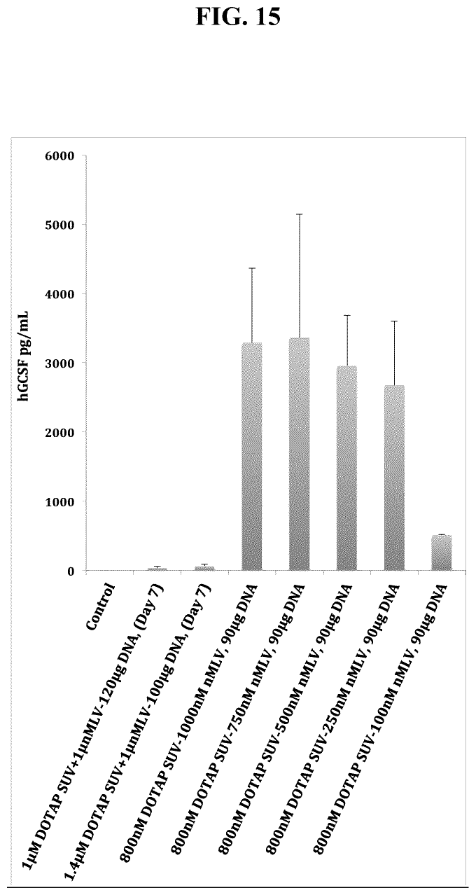

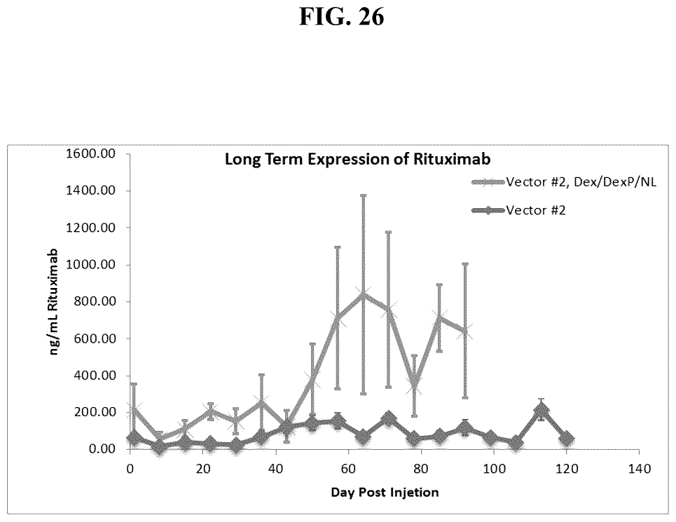

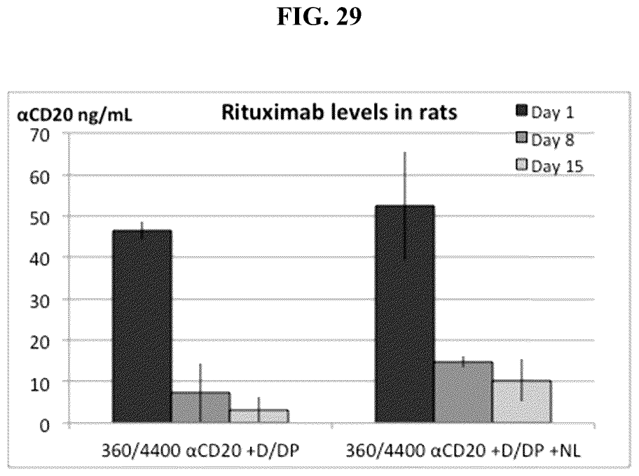

Fifth, as demonstrated in example 14, the addition of neutral lipids together with dexamethasone and dexamethasone palmitate can increase the duration of gene expression (FIGS. 26 and 29). In contrast, the administration of neutral lipid alone can decrease the duration of gene expression (FIGS. 14 and 15).

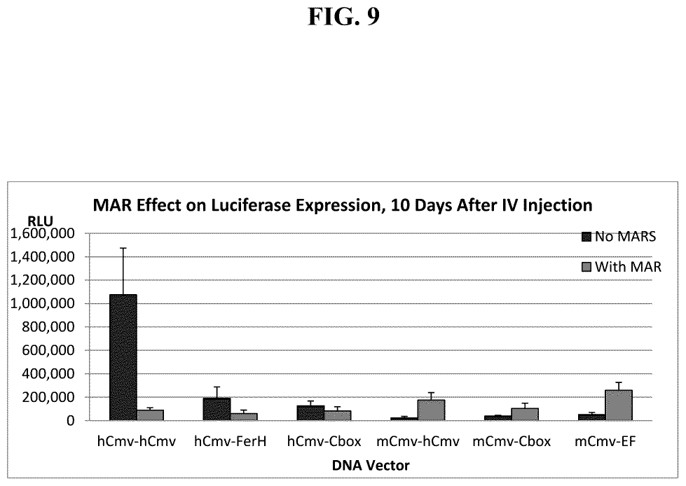

In addition, some literature also describes that matrix attachment regions (MAR) should be incorporated into DNA vectors in order to produce prolonged expression following their IV injection (Argyros et al., J Mol Med (2011) 89:515-529, herein incorporated by reference in its entirety). In contrast, work conducted during development of embodiments of the present disclosure indicate that the presence of such MAR elements do not increase, and in some vectors decrease the duration of gene expression produced by IV, sequential injection of cationic liposomes followed by CPG-free plasmid DNA (FIG. 9).

In certain embodiments, the present disclosure employs polycationic structures (e.g., empty cationic liposomes, empty cationic micelles, or empty cationic emulsions) not containing vector DNA, which are administered to a subject prior to vector administration. In certain embodiments, the polycationic structures are cationic lipids and/or are provided as an emulsion. The present disclosure is not limited to the cationic lipids employed, which can be composed, in some embodiments, of one or more of the following: DDAB, dimethyldioctadecyl ammonium bromide; DPTAP (1,2-dipalmitoyl 3-trimethylammonium propane); DHA; prostaglandin, N-[1-(2,3-Dioloyloxy) propyl]-N,N,N-trimethylammonium methylsulfate; 1,2-diacyl-3-trimethylammonium-propanes, (including but not limited to, dioleoyl (DOTAP), dimyristoyl, dipalmitoyl, disearoyl); 1,2-diacyl-3-dimethylammonium-propanes, (including but not limited to, dioleoyl, dimyristoyl, dipalmitoyl, disearoyl) DOTMA, N-[1-[2,3-bis(oleoyloxy)]propyl]-N,N,N-trimethylammonium chloride; DOGS, dioctadecylamidoglycylspermine; DC-cholesterol, 3.beta.-[N-(N′,N′-dimethylaminoethane) carbamoyl]cholesterol; DOSPA, 2,3-dioleoyloxy-N-(2 (sperminecarboxamido)-ethyl)-N,N-dimethyl-1-propanami-nium trifluoroacetate; 1,2-diacyl-sn-glycero-3-ethylphosphocholines (including but not limited to dioleoyl (DOEPC), dilauroyl, dimyristoyl, dipalmitoyl, distearoyl, palmitoyl-oleoyl); beta-alanyl cholesterol; CTAB, cetyl trimethyl ammonium bromide; diC14-amidine, N-t-butyl-N′-tetradecyl-3-tetradecylaminopropionamidine; 14Dea2, O,O′-ditetradecanolyl-N-(trimethylammonioacetyl) diethanolamine chloride; DOSPER, 1,3-dioleoyloxy-2-(6-carboxy-spermyl)-propylamide; N,N,N′,N′-tetramethyl-N,N′-bis(2-hydroxylethyl)-2,3-dioleoyloxy-1,4-butan-ediammonium iodide; 1-[2-acyloxy)ethyl]2-alkyl (alkenyl)-3-(2-hydroxyethyl-) imidazolinium chloride derivatives such as 1-[2-(9 (Z)-octadecenoyloxy) eth-yl]-2-(8 (Z)-heptadecenyl-3-(2-hydroxyethyl) imidazolinium chloride (DOTIM), 1-[2-(hexadecanoyloxy)ethyl]-2-pentadecyl-3-(2-hydroxyethyl) imidazolinium chloride (DPTIM); 1-[2-tetradecanoyloxy)ethyl]-2-tridecyl-3-(2-hydroxyeth-yl) imidazolium chloride (DMTIM) (e.g., as described in Solodin et al. (1995) Biochem. 43:13537-13544, herein incorporated by reference); 2,3-dialkyloxypropyl quaternary ammonium compound derivates, containing a hydroxyalkyl moiety on the quaternary amine, such as 1,2-dioleoyl-3-dimethyl-hydroxyethyl ammonium bromide (DORI); 1,2-dioleyloxypropyl-3-dimethyl-hydroxyethyl ammonium bromide (DORIE); 1,2-dioleyloxypropyl-3-dimethyl-hydroxypropyl ammonium bromide (DORIE-HP), 1,2-dioleyloxypropyl-3-dimethyl-hydroxybutyl ammonium bromide (DORIE-HB); 1,2-dioleyloxypropyl-3-dimethyl-hydroxypentyl ammonium bromide (DORIE-HPe); 1,2-dimyristyloxypropyl-3-dimethyl-hydroxylethyl ammonium bromide (DMRIE); 1,2-dipalmityloxypropyl-3-dimethyl-hydroxyethyl ammonium bromide (DPRIE); 1,2-disteryloxypropyl-3-dimethyl-hydroxyethyl ammonium bromide (DSRIE) (e.g., as described in Felgner et al. (1994) J. Biol. Chem. 269:2550-2561, herein incorporated by reference in its entirety). Many of the above-mentioned lipids are available commercially from, e.g., Avanti Polar Lipids, Inc.; Sigma Chemical Co.; Molecular Probes, Inc.; Northern Lipids, Inc.; Roche Molecular Biochemicals; and Promega Corp.

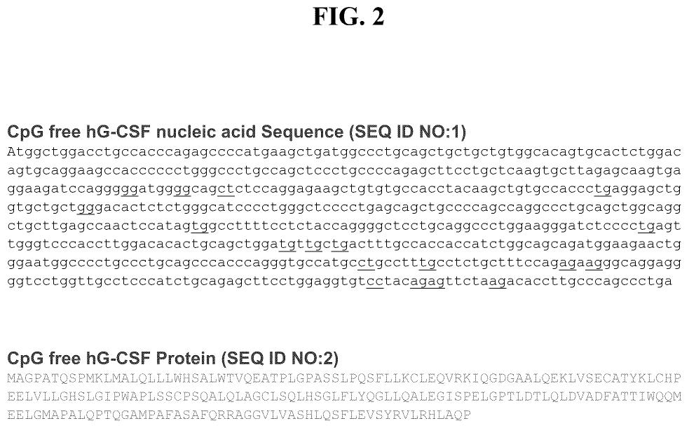

In certain embodiments, the present disclosure employs CpG-reduced or CpG-free expression vectors. An initial sequence that contains CpG dinucleotides (e.g., wild-type version of human G-CSF), may be modified to remove CpG dinucleotides by altering the nucleic acid sequence. FIG. 2 shows a CpG-free version of human G-CSF, with sequences that have been changed to removed CpGs underlined. Such CpG di-nucleotides can be suitably reduced or eliminated not just in a coding sequence, but also in the non-coding sequences, including, e.g., 5′ and 3′ untranslated regions (UTRs), promoter, enhancer, polyA, ITRs, introns, and any other sequences present in the nucleic acid molecule or vector. CpG di-nucleotides may be located within a codon triplet for a selected amino acid. There are five amino acids (serine, proline, threonine, alanine, and arginine) which have one or more codon triplets that contain a CpG di-nucleotide. All five of these amino acids have alternative codons not containing a CpG di-nucleotide that can be changed to, to avoid the CpG but still code for the same amino acid as shown in Table 1 below. Therefore, the CpG di-nucleotides allocated within a codon triplet for a selected amino acid may be changed to a codon triplet for the same amino acid lacking a CpG di-nucleotide.

TABLE 1

| | DNA Codons | DNA Codons |
|---|---|---|
| Amino Acid | Containing CpG | Lacking CpG |
| Serine (Ser or S) | TCG | TCT, TCC, TCA, |
| | | AGT, AGC |
| Proline (Pro or P) | CCG | CCT, CCC, CCA, |
| Threonine (Thr or T) | ACG | ACA, ACT, ACC |
| Alanine (Ala or A) | GCG | GCT, GCC, GCA |
| Arginine (Arg or R) | CGT, CGC, | AGA, AGG |
| | CGA, CGG | |

In addition, within the coding region, the interface between triplets should be taken into consideration. For example, if an amino acid triplet ends in a C-nucleotide which is then followed by an amino acid triplet which can start only with a G-nucleotide (e.g., Valine, Glycine, Glutamic Acid, Alanine, Aspartic Acid), then the triplet for the first amino acid triplet is changed to one which does not end in a C-nucleotide. Methods for making CpG sequences are shown, for example, in U.S. Pat. No. 7,244,609, which is herein incorporated by reference. A commercial service provided by INVIVOGEN is also available to produce CpG free (or reduced) nucleic acid sequences and vectors.

Provided below in Table 2 are exemplary promoters and enhancers that may be used in the vectors described herein. Such promoters, and other promoters known in the art, may be used alone or with any of the enhancers, or enhancers, known in the art. Additionally, when multiple proteins or biologically active nucleic acid molecules (e.g., two, three, four, or more) are expressed from the same vector, the same or different promoters may be used in conjunction with the subject nucleic acid sequence.

TABLE 2

| Promoter | Enhancer |
|---|---|
| CMV | human CMV |
| EF1α | mouse CMV |
| Ferritin (Heavy/Light) Chain | SV40 |
| GRP94 | Ubc |
| U1 | AP1 |
| UbC | hr3 |
| Beta Actin | IE2 |
| PGK1 | IE6 |
| GRP78 | E2-RS |
| CAG | MEF2 |
| SV40 | C/EBP |
| TRE | HNF-1 |

The present disclosure is not limited by the type of therapeutic proteins that is expressed. In certain embodiments, the therapeutic protein comprises an antibody or antibody fragments (e.g., F (ab) or F (ab′) 2). In other embodiments, the therapeutic protein is selected from the group consisting of an anti-inflammatory protein, coagulation protein, anti-cancer protein, anti-sepsis protein, etc.

### EXAMPLES

### Example 1

### In Vivo Protein Expression Using Sequential Injection of Cationic Liposomes Followed by CPG-Free Expression Vectors

This example describes various work using in vivo protein expression using sequential injection of cationic liposomes followed closely in time by CPG-free expression vectors.

### Methods

Liposome preparation. Pure DOTAP lipid as a lyophilized powder was purchased from Avanti polar lipids. Pure DOTAP cationic liposomes were prepared by re-suspending the lyophilized powder in a solution of 5% dextrose in water at a lipid concentration of 20 millimolar. The solution was then vortexed for 15 minutes to form multi-lamellar vesicles (MLV), mean particle size 350 nm, as measured by laser light scattering. Small uni-lamellar vesicles (SUV), mean particle size 75 nm, were then formed from MLV by sonication in a bath sonicator.
Plasmid construction. General schematics for the vectors employed are provided in FIG. 1. In general, a CpG free DNA plasmid vector is typically composed of the following elements: enhancer/promoter/5′UTR of either mCMV/EF1/I126 (851 bp) or hCMV/hCMV/HTLV (873 bp), linked to a gene of interest (such as h-GCSF (615 bp) or soLux (1653 bp)), minimal polyA (63 bp), MARs derived from either BGlobin (434 bp), 21q21 (1055 bp) or IFN (820 bp) and an R6K Ori/Kan^{r} (Kanamycin antibiotic resistant) expression cassette (1206 bp). R6Kori/Kan^{r} DNA was designed as a base vector containing three endonuclease restriction enzyme sites, DraIII, EcoRI and NheI. It was assembled from gBlock four DNA fragments (IDT, IA) using the Gibson Assembly technique (NEB, MA). For MAR containing plasmids, a BGlobin MAR was inserted into the base vector at DraIII-EcoRI sites. The CpG-free nucleic acid sequence for h-GCSF is shown in FIG. 2.

The expression cassette was constructed using the puc19 plasmid backbone by sequentially inserting each DNA element between EcoRI and XbaI. Enhancer/Promoter elements containing 5′ EcoRI and NheI sites were ligated to the 5′UTR, gene, pA or pA-MAR, as well as puc19 at EcoRI, EcoRV, BstEII, BgIII and XbaI sites, respectively. The expression cassette was then digested with EcoRI-XbaI and inserted into the base vector at EcoRI-NheI, producing an expression plasmid containing restriction sites that can be used to insert a second expression cassette insertion. Dual (Luc-and GCSF) cassette expression plasmids were then constructed by inserting the hG-CSF expression cassette into the base vector at EcoRI-NheI. The second, Luc expression cassette was subsequently inserted into the G-CSF expression plasmid at EcoRI-NheI, producing a dual cassette, Luc and GCSF containing, single plasmid vector.

Plasmid Purification. Endotoxin-free plasmids were purified on 5′Prime Endo-free Maxi columns as follows. Briefly, 200 ml of bacteria containing the plasmid are grown overnight at 37 C and then collected. Bacterial cells are lysed per the manufacturer's protocol. Endotoxin is removed using an EndoFree filter CS. Isopropanol is added to the lysate and then loaded onto a column. After successive washes, the column is centrifuged and air-dried for 10 min to ensure residual ethanol is removed. DNA is then eluted from the column with 1 ml of Lactated Ringers.
Mice. 21 g female, CD-1 mice were purchased from Charles River. Housing, care and all procedures were performed according to IACUC approved guidelines.
Sequential injection of cationic liposomes, then plasmid DNA in mice. Three to five mice were injected per group. Each mouse received a single IV injection of cationic liposomes (MLV or SUV), followed two minutes later by a single IV injection of a CPG-free, plasmid DNA vector.
Obtaining and then analyzing mouse serum for human G-CSF levels. Each mouse was anesthetized and then bled via the submandibular vein. Serum was then isolated from whole blood and human G-CSF levels measured in pg/ml, as performed strictly according to the manufacturer's specifications, using an R and D systems human G-CSF ELISA.
Obtaining and then analyzing mouse tissue for luciferase activity. Lung was homogenized with 500 ul of 1× Lysis buffer (Promega, WI). The homogenate was centrifuged at 3000×g at 4 C for 10 min. and the supernatant collected. Luciferase activity was assayed using 20 ul of supernatant and 100 μl of Luciferase reagent for 10 seconds using a GloMax® Luminometer (Promega, WI).

### Results/Description

Serum human G-CSF levels produced in mice by sequential, IV cationic liposome injection followed by IV DNA vector injection

Five mice were injected per group. Each mouse received a single IV injection of 800 nmoles of pure DOTAP cationic liposomes (MLV or SUV), followed two minutes later by a single IV injection of 80 ug of an mCMV-EF1-hGCSF, an hCMV-hCMV-hGCSF or an mCMV-EF1-luciferase, CPG-free, plasmid DNA vector. Serum hG-CSF levels were assessed beginning at day seven after IV injection, and at seven-day intervals thereafter.

As shown in FIG. 3, all three DNA vectors containing the hG-CSF gene produced supra-therapeutic hG-CSF levels (≥100 pg/ml is required to increase neutrophil levels), at day seven after injection. Thereafter, hG-CSF levels rose progressively until day 21, and then remained stable until day 63, the last time point analyzed. In contrast, hG-CSF levels produced by identical, IV sequential injection of the EF1-luciferase DNA vector were undetectable throughout the course of the experiment.

WBC and Absolute Neutrophil Counts, 21 Days after Sequential, IV Cationic Liposome then DNA Vector Injection

Whole blood was collected from groups of 4 mice at day 21 following sequential, IV injection of DOTAP MLV, followed two minutes later by a single IV injection of either an EF1-hGCSF or an EF-1 luciferase-containing, CPG-free plasmid DNA vector. Blood from each mouse was then analyzed, in a blinded fashion, for total WBC, as well as absolute neutrophil counts by the University of California Davis veterinary diagnostics laboratory.

As shown in FIG. 4, one sequential IV injection of DOTAP cationic liposomes followed by IV injection of EF1-hG-CSF DNA increased absolute neutrophil counts approximately 10 fold and total WBC approximately 4 fold, 21 days following injection when compared to mock-injected control mice receiving sequential injection of an EF1-luciferase, plasmid DNA vector. These results document that the hG-CSF gene encoded protein product was fully functional in treated mice. Taken together, the high-level increases in absolute neutrophil counts produced by the EF1-hG-CSF DNA vector, coupled with the 10 to 15 fold above therapeutic hG-CSF protein levels produced at day 21 (see FIG. 3) demonstrate that a single, sequential IV injection of a cationic liposomes followed by a CPG-free DNA vector can produce prolonged therapeutic effects of a now FDA-approved recombinant human protein therapy.

Serum Human G-CSF Levels Produced in Mice by Sequential IV Injection of Either Single or Dual Cassette, hG-CSF Single Plasmid Vectors

Sera were collected from groups of four mice at either day 6 or 14 following sequential, IV injection of 800 nmoles of pure DOTAP MLV cationic liposomes, followed two minutes later by IV injection of 40 ug either an EF1-luciferase-EF1-hGCSF (2 expression cassette) or an EF1-hGCSF (1 expression cassette), CPG-free, single plasmid DNA vector.

As shown in FIG. 5, the single as well as dual cassette DNA vectors containing the hG-CSF gene each produced supra-therapeutic serum hG-CSF levels (≥100 pg/ml required to increase neutrophil counts) at day six after injection, 951 and 423 pg/ml, respectively. The single cassette vector produced even higher therapeutic levels, 1941 pg/ml, at day 14. In contrast, hG-CSF levels produced at day 14 by the dual cassette, single plasmid DNA vector had fallen to a sub-therapeutic level (93 pg/ml), as hG-CSF protein levels below 100 pg/ml are sub-therapeutic. Thus, adding a second expression cassette can control the duration of expression of the gene contained in the first cassette.

Serum hG-CSF Levels in Mice, 21 Days after IV Injection of Cationic Liposomes, then DNA Containing Different Promoter-Enhancer Combinations Linked to the hG-CSF Gene

Sera were collected from groups of four mice, 21 days following sequential, IV injection of 800 nmoles of pure DOTAP MLV cationic liposomes, followed two minutes later by IV injection of 60 ug of the hG-CSF gene, linked to one of the following enhancer-promoter combinations, mCMV-EF1, hCMV-hCMV, hCMV-hferritin light chain, hCMV-hferritin heavy chain, hCMV-glucose-regulated protein 78 or mCMV-hferritin light chain, each in a CPG-free, single cassette, DNA vector.

As shown in FIG. 6, a range of supra-therapeutic, hG-CSF serum levels were produced at day 21 by the mCMV-EF-1 (2120 pg/ml), hCMV-hCMV (1516 pg/ml), hCMV-FerL (699 pg/ml), hCMV-Grp78 (343 pg/ml) and mCMV-FerL (303 pg/ml)-driven DNA vectors, each linked to the hG-CSF gene. In contrast, the hCMV-FerH-hG-CSF DNA vector (52 pg/ml) produced a sub-therapeutic hG-CSF level. Taken together, these results reveal that changing the promoter-enhancer combination can produce a range of different hG-CSF protein levels, from more than 20 fold above therapeutic to sub therapeutic, 21 days after a single injection. (hG-CSF protein levels ≥100 pg/ml are required to increase neutrophil counts).

Mouse Lung Luciferase Levels, 7 Days after Sequential IV Injection of Cationic Liposomes, then Single Cassette, EF1-Luciferase DNA Alone or Together with a Drug

Lungs were collected from groups of four mice, 7 days following sequential, IV injection of 800 nmoles of pure DOTAP MLV cationic liposomes alone, or containing 2 mg/kg of L-arginine, 0.01 mg/kg of colchicine or 1 mg/kg of dexamethasone. In each case, cationic liposome injection was followed two minutes later by IV injection of 40 ug of an mCMV-EF1-luciferase, CPG-free, single cassette, DNA vector.

As shown in FIG. 7, when compared to mice receiving sequential injection of DOTAP MLV alone (control), mice receiving either colchicine or dexamethasone together with the liposomes showed higher luciferase activity in the lung. In contrast, mice receiving L-arginine together with liposomes failed to increase gene expression levels. Thus, co-injecting selected drugs together with the liposomes can increase the level and duration of expression of genes delivered by sequential cationic liposome then DNA injection.

Mouse Lung Luciferase Levels, 7 Days after Sequential IV Injection of Cationic Liposomes, then Dual Cassette, EF1-hG-CSF-EF1-Luciferase DNA Alone or with Drug(s)

Lungs were collected from groups of four mice, 7 days following sequential, IV injection of 800 nmoles of pure DOTAP MLV cationic liposomes alone, or containing 2 mg/kg of L-arginine, 1 mg/kg of dexamethasone, 0.02 mg/kg of sildenafil, 0.1 mg/kg of valproic acid or 2 mg/kg of L-arginine plus 0.02 mg/kg of sildenafil (VIAGRA). In each case, cationic liposome injection was followed two minutes later by IV injection of 40 ug of EF1-luciferase-EF1-hGCSF, a 2 expression cassette, CPG-free single plasmid DNA vector.

As shown in FIG. 8, when compared to mice receiving sequential injection of DOTAP MLV alone (control), mice receiving dexamethasone, valproic acid or sildenafil alone, or L-arginine plus sildenafil together with the liposomes showed higher luciferase activity in the lung. In contrast, mice receiving either L-arginine or valproic acid together with liposomes were either lower than or comparable to controls. Thus, depending on the drug co-injected, expression levels of the delivered gene can be increased or reduced.

Mouse Lung Luciferase Levels, 10 Days after Sequential IV Injection of Cationic Liposomes, then a DNA Vector Containing One of a Series of Different Promoter-Enhancer Combinations, Each Either with or without MARs and all Linked to the Luciferase Gene

Lungs were collected from groups of three mice, 10 days following sequential, IV injection of 800 nmoles of pure DOTAP MLV cationic liposomes, followed two minutes later by IV injection of 40 ug of the luciferase gene, linked to one of the following enhancer-promoter combinations: hCMV-hCMV, hCMV-human ferritin heavy chain, hCMV-CBOX (human Carboxypeptidase B1), mCMV-hCMV, mCMV-CBOX and mCMV-EF1, each linked to the luciferase gene in a CPG-free, single cassette, DNA vector.

FIG. 9 shows that DNA vectors lacking MARs, and containing the hCMV enhancer linked to the hCMV, ferritin heavy chain or CBOX promoters produced higher lung luciferase levels than the corresponding vectors containing MAR elements. In contrast, DNA vectors containing both MARs and the mCMV enhancer linked to the hCMV, EF1 or CBOX promoters failed to produce lung luciferase levels as high as the corresponding vectors lacking MAR elements. Thus, CPG-free DNA vectors lacking MARs can produce more durable expression than MAR-containing vectors.

Mouse Lung Luciferase Levels, 1 or 5 Days after IV Injection of PEI:EF-1 Luc DNA Complexes or Sequential IV Injection of Cationic Liposomes, then the Identical EF-1 Luc DNA

Lungs were collected from groups of three mice, 1 or 5 days following IV injection of either 12.5 ug of CPG-free EF-1-Luc DNA vector complexed to 22 kDa linear PEI at a 1:4 N:P ratio, or sequential, IV injection of 900 nmoles of pure DOTAP MLV cationic liposomes, followed two minutes later by IV injection of 40 ug of the same EF-1-Luc DNA vector.

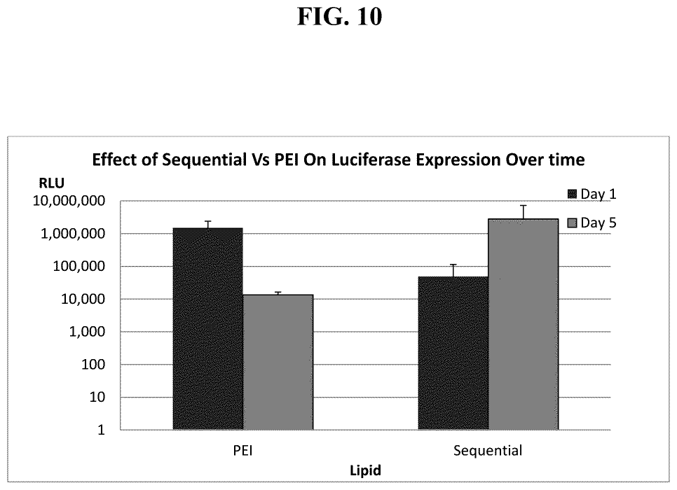

FIG. 10 shows lung luciferase levels were consistently higher in the PEI:DNA injected mice than in the mice injected sequentially with cationic liposomes then DNA at day one following injection. However, lung luciferase levels had fallen approximately 100 fold in the PEI:DNA injected mice by day five. In direct contrast, lung luciferase levels from the sequentially IV injected mice had risen by day 5 after injection. Luciferase levels were up to 150 fold higher in sequentially-injected mice than those present in PEI:DNA injected mice also sacrificed at day 5. Thus, sequential cationic liposome then DNA injection produces higher levels of gene expression at later time points when compared to the same CPG free DNA injected as a PEI:DNA complex.

Mouse Lung Luciferase Levels, 1 Day after Sequential IV Injection of One of Seven Different Cationic Liposome Formulations, then Single Cassette, EF1-Luciferase DNA.

Lungs were collected from groups of four mice, 1 day following sequential, IV injection of 800 nmoles of pure DOTAP MLV, pure DOTAP SUV, DOTAP:cholesterol 2:1 ML V, DOTAP:diolelyl phosphatidylcholine (DOPC) 1:1 MLV, DSTAP MLV, ethyl DSPC MLV or DOTAP:DOBAQ 1:1 MLV cationic liposomes. In each case, cationic liposome injection was followed two minutes later by IV injection of 80 ug of an mCMV-EF1-luciferase, CPG-free, single cassette, DNA vector.

As shown in FIG. 11, when compared to lung luciferase levels in mice receiving sequential IV injection of pure DOTAP MLV, mice receiving DOTAP SUV, DOTAP:chol or DOTAP:DOPC cationic liposome formulations produced gene expression levels approximating DOTAP MLV or higher. In contrast, mice receiving DSTAP, ethyl DSPC or DOTAP:DOBAQ MLV produced either very low or nearly undetectable lung luciferase levels. That DOTAP SUV produced gene expression levels approximating DOTAP MLV was unexpected because DOTAP MLV produces more than 1700 fold higher levels of gene expression than DOTAP SUV when injected as cationic liposome:DNA complexes (see, Nature Biotechnology, 15:167-173; 1996, herein incorporated by reference in its entirety).

Mouse Spleen Luciferase Levels, 1 Day after IV Injection of PEI:EF-1 Luc DNA Complexes Alone or Mixed with One of Four Different Drugs.

Spleens were collected from groups of three mice, 1 day following IV injection of 12.5 ug of CPG-free EF-1-Luc DNA vector complexed to 22 kDa linear PEI at a 1:4 N:P ratio. Mice received an intraperitoneal injection of one ml of 5% DMSO either alone, or containing 200 ug of amlexanox, 1 mg of chloroquine, 200 ug of SAHA or 300 ug of tofacitinib per mouse, two hours prior to receiving IV PEI:DNA complexes.

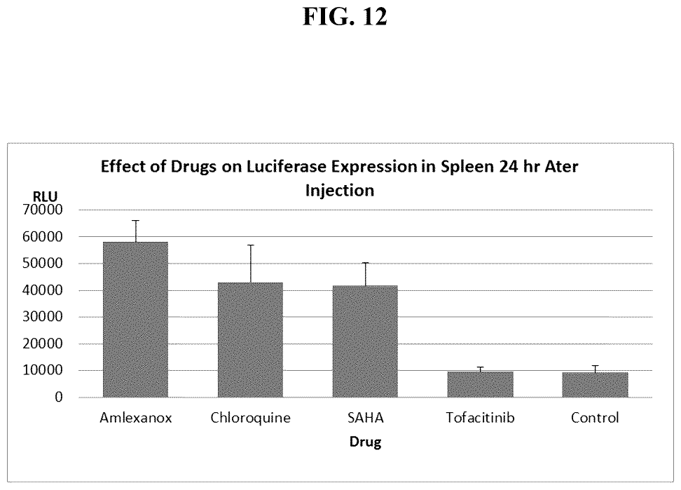

FIG. 12 shows pre-injection of the anti-inflammatory agents amlexanox, chloroquine or SAHA prior to injecting CPG-free DNA increased gene expression levels, whereas tofacitinib failed to increase gene expression. Amlexanox in particular, a selective inhibitor of the TBK1-induced interferon activation pathway, increased gene expression levels. Thus, pre-injection of selected anti-inflammatory agents may further increase the effectiveness of CPG-free DNA for gene therapy.

Mouse Lung Luciferase Levels, 1 or 7 Days after Sequential IV Injection of Cationic Liposomes, then One of a Series of Dual Cassette, EF-1-Luc-hG-CSF DNA Vectors

Lungs were collected from groups of four mice, 1 or 7 days following sequential, IV injection of 800 nmoles of pure DOTAP MLV cationic liposomes followed two minutes later by 40 μg of EF1-Luc-EF1-hGCSF, EF1-Luc-hCMV-hCMV-hGCSF, EF1-Luc-hCMV-hCBOX-hGCSF, EF1-Luc-hCMV-hREG1-hGCSF or EF1-Luc-mCMV-hCBOX-hGCSF 2 expression cassette, CPG-free single plasmid DNA vector.

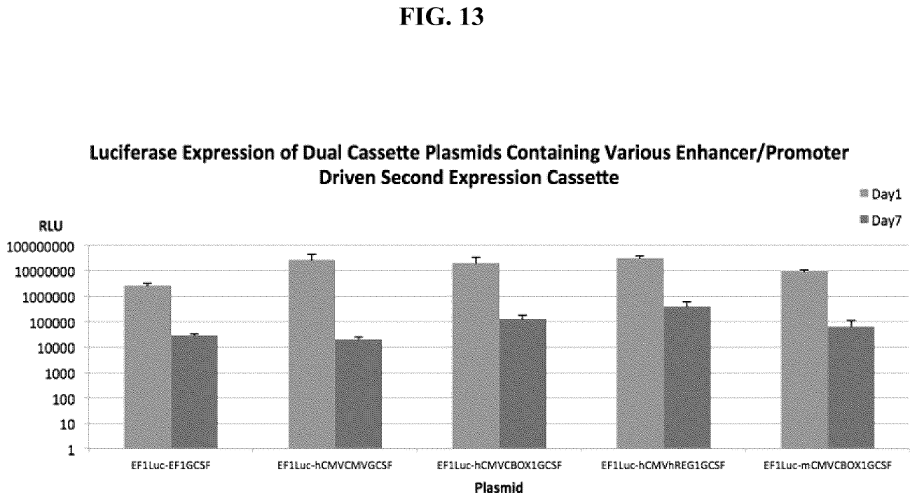

FIG. 13 shows that when compared to mice receiving sequential injection of EF1-Luc-EF1-hGCSF dual cassette vector, containing the EF1 promoter in each cassette (control), mice receiving each of the other four dual cassette DNA vectors containing the EF1 promoter in one cassette and another promoter in the second cassette showed consistently higher luciferase activity in the lung at both day one and seven after injection. Dual cassette vectors containing a different promoter in each cassette produced lung luciferase levels up to tenfold or more higher at each time point than produced by the dual cassette vector containing the EF-1 promoter in both cassettes. Thus, using different promoter elements in different cassettes of multi-cassette vectors can significantly increase the level and duration of gene expression they produce.

Serum Human G-CSF Levels Produced in Mice, 1 Day after Sequential, IV Cationic Liposome Injection, with or without Co-Injection of Neutral Liposomes, Followed by IV Injection of a Dual Cassette, Single Plasmid Vector.

Neutral MLV liposomes were prepared from Phospholipon 90H, Lipoid GmbH. The fatty acid content of this product is 15% palmitic acid, 85% stearic acid. Liposomes were prepared either by drying down the lipids in organic solvent on a rotary evaporator, then re-suspending the dried lipid film in a solution of 5% dextrose in water at a lipid concentration of 50 millimolar or by hydrating the lipid as a dry powder in 5% w/v dextrose. Both were prepared at 60 degrees C. The solution was then vortexed for 15 minutes to form MLV. sera were collected from groups of four mice at day 1 following sequential, IV injection of buffer alone (control), 1000 nmoles of pure DOTAP SUV cationic liposomes, alone or co-injected with 1000 nmol of neutral MLV, followed two minutes later by IV injection of 120 μg an EF1-luciferase—EF1-hGCSF (2 expression cassette), CPG-free, single plasmid DNA vector or 1400 nmoles of pure DOTAP SUV cationic liposomes, alone or co-injected with 1000 nmol of neutral MLV, followed two minutes later by IV injection of 100 μg of EF1-luciferase—EF1-hGCSF DNA.

As shown in FIG. 14, serum hG-CSF levels produced one day after sequentially co-injecting pure DOTAP SUV together with neutral MLV then EF1-luciferase-EF1-hGCSF DNA were increased from 3 to 600 fold when compared to sequential injection of DOTAP SUV without neutral MLV. Thus, co-injecting neutral liposomes together with cationic liposomes can significantly increase peak levels of gene expression produced. In addition, co-injecting neutral liposomes appears to eliminate the variation in gene expression levels produced by sequentially injecting different ratios of cationic liposomes to DNA without co-injecting neutral liposomes.

Serum Human G-CSF Levels Produced in Mice, 1 or 7 Days after Sequential, IV Cationic Liposome Co-Injection with Neutral Liposomes, Followed by IV Injection of a Dual Cassette Plasmid Vector

Sera were collected from groups of four mice at day 7 following sequential, IV injection of 1000 nmoles pure DOTAP SUV cationic liposomes co-injected with 1000 nmol of neutral MLV, followed two minutes later by IV injection of 120 μg of CPG-free, EF1-luciferase-EF1-hGCSF DNA or 1000 nmoles of pure DOTAP SUV cationic liposomes co-injected with 1400 nmol of neutral MLV, followed two minutes later by IV injection of 100 μg of EF1-luciferase-EF1-hGCSF DNA. Sera were also collected from groups of four mice at day 1 following sequential, IV injection of buffer only (control), 800 nmoles of pure DOTAP SUV cationic liposomes co-injected with 1000, 750, 500, 250 or 100 nmol of neutral MLV respectively, followed two minutes later by IV injection of 90 μg of EF1-luciferase-EF1-hGCSF.

As shown in FIG. 15, serum hG-CSF levels produced by co-injecting pure DOTAP SUV together with neutral MLV dropped by approximately 100 fold compared to the hG-CSF levels produced in the same mice one day after injection, (See FIG. 14 for hG-CSF levels produced at day 1 following injection of these same mice). Thus, co-injecting neutral liposomes with cationic liposomes can strongly alter both peak as well as longer-term expression of delivered genes. In addition, the ratio of neutral to cationic liposomes co-injected determines the extent to which co-injected neutral liposomes increase the expression of sequentially delivered genes.

Mouse Serum hG-CSF Levels, 7 Day after Sequential IV Injection of Different Cationic Liposome Formulations, then Single Cassette, CPG-Free EF1-hG-CSF DNA.

Sera were collected from groups of four mice, 7 days following sequential, IV injection of either 800 or 1000 nmoles of pure DOTAP SUV, or MLV cationic liposomes. Injection of each of the three cationic liposome formulations was followed two minutes later by IV injection of either 100 or 120 μg of an mCMV-EF1-h-G-CSF, CPG-free, single cassette, DNA vector. 0.1 μm extruded cationic liposomes were prepared from MLV by sequential extrusion through sized polycarbonate membranes under high argon gas pressure in a Lipex extrusion device.

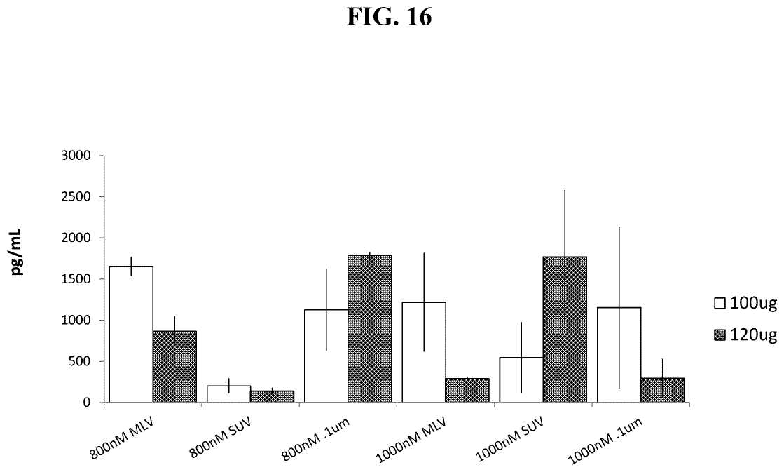

As shown in FIG. 16, and in part depending on the ratio of nmoles cationic liposomes to μg DNA ratio injected, pure DOTAP SUV and 0.1 μm extruded cationic liposomes produced extended, high-level expression of hG-CSF as efficiently as that produced by MLV cationic liposomes. Therefore, SUV as well as 0.1 μm extruded (oligolamellar) cationic liposomes are as effective as MLV when used for sequential cationic liposome then CPG-free DNA injection.

### Example 2

### Long-Term Expression

In this Example, five mice per group were sequentially injected with 800 nmol of either DOTAP MLV or SUV followed by 90 μg of a CPG-free plasmid vector containing an EF1- or hCMV-driven hG-CSF cDNA. Serum levels of human G-CSF protein were assessed at 7- or 14-day intervals for the subsequent 428 days following injection. Obtaining and analyzing mouse serum for human G-CSF levels was performed as follows. Each mouse was anesthetized and bled via submandibular vein. Serum was isolated from whole blood using serum separator tubes from BD. Human G-CSF levels were measured in pg/ml via an ELISA performed strictly according to the manufacturer's specifications, using an R&D systems human G-CSF ELISA. The results are shown in FIG. 17, and show that supra-therapeutic levels of human G-CSF protein were produced in fully immune-competent mice for at least 428 days after receiving a single IV injection of DOTAP SUV liposomes then an EF1-huG-CSF plasmid DNA vector.

### Example 3

### Protein Expression in Rats

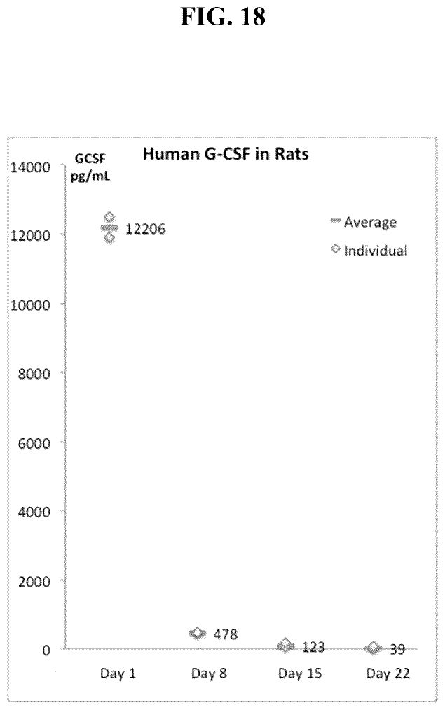

In this Example, 250 gm Sprague-Dawley female rats #22 and 23 were sequentially injected with 6000 nmol of DOTAP SUV then 300 μg of a CPG-free plasmid vector containing an EF1-driven hG-CSF DNA vector. Serum levels of human G-CSF protein, WBC and absolute neutrophil counts (ANC) were assessed at 7-day intervals following injection. Serum ALT and AST levels were assessed at day 1 only. All were assessed by the UC Davis Comparative Pathology lab. As shown in FIG. 18, and Table 3 below, supra-therapeutic levels of hG-CSF protein, as well as significantly elevated WBC and ANC, were produced in EF1-huG-CSF injected rats for at least 22 days following a single IV injection. ALT and AST measured at day 1 after injection were comparable to background control levels in un-injected rats.

TABLE 3

| WBC | Day 1 | Day 8 | Day 15 | Day 22 | | | ALT | Day 1 | | |
|---|---|---|---|---|---|---|---|---|---|---|
| #22 | 6.56 | 27.36 | 23.68 | 27.20 | Ctrl | 4.57 | #22 | 30.30 | Ctrl | 12-67 |
| #23 | 11.04 | 15.74 | 12.90 | 20.08 | SEM | 0.27 | #23 | 29.40 | | |
| ANC | Day 1 | Day 8 | Day 15 | Day 22 | | | AST | Day 1 | | |
| #22 | 4.99 | 17.31 | 12.32 | 15.69 | Ctrl | 1.39 | #22 | 89.80 | Ctrl | 14-113 |
| #23 | 5.07 | 8.07 | 6.68 | 10.57 | SEM | 0.13 | #23 | 74.00 | | |

### Example 4

### DPTAP Liposomes

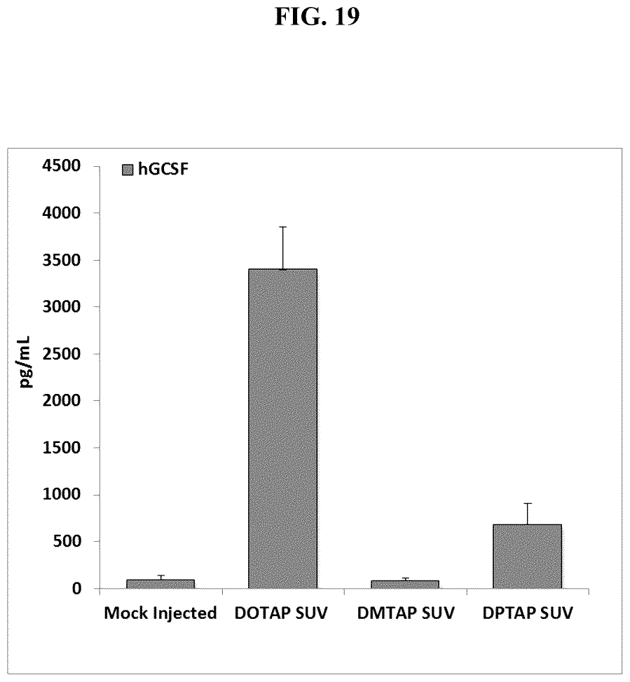

In this Example, three mice were injected per group. Each mouse received a single IV injection of 900 nmoles of DOTAP, DMTAP, or DPTAP (1,2-dipalmitoyl 3-trimethylammonium propane) SUV liposomes followed two minutes later by a single IV injection of 70 μg of an EF-1 plasmid DNA vector encoding hG-CSF. Serum levels of hG-CSF were determined by ELISA 24 hours following injection. FIG. 19 shows that HuG-CSF protein was present in serum from mice treated with DOTAP or DPTAP but not DMTAP. These data indicate that multiple cationic lipids can mediate transfection in vivo, and that level of protein production can be controlled by selection of the lipid carrier.

### Example 5

### Toxicity Resolves within 48 Hours

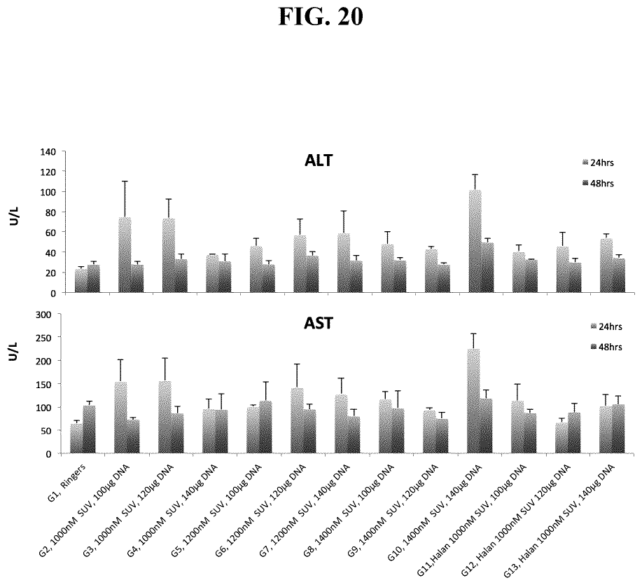

In this Example, three mice were injected per group. Mice were purchased from Charles River Labs. Each mouse received a single IV injection of 1000 nmoles, 1200 nmoles, or 1400 nmoles of DOTAP SUV liposomes as indicated, followed two minutes later by a single IV injection of 100 μg, 120 μg, or 140 μg of a CPG-free EF-1 driven plasmid DNA vector encoding luciferase. Serum was collected at 24 hrs or 48 hrs after injection. ALT and AST measurements were assayed at the UC Davis Comparative Pathology lab. As shown in FIG. 20, at 24 hours following sequential injection, serum levels of ALT and AST were elevated from two to five fold in all lipid then DNA groups. At 48 hours, serum ALT and AST levels returned to control (background) levels (shown by the mock-injected group). These data indicate that toxicity as measured by ALT/AST is acute (present within 24 hrs of injection) and transient (gone by 48 hrs).

### Example 6

### Liposomes with DexP

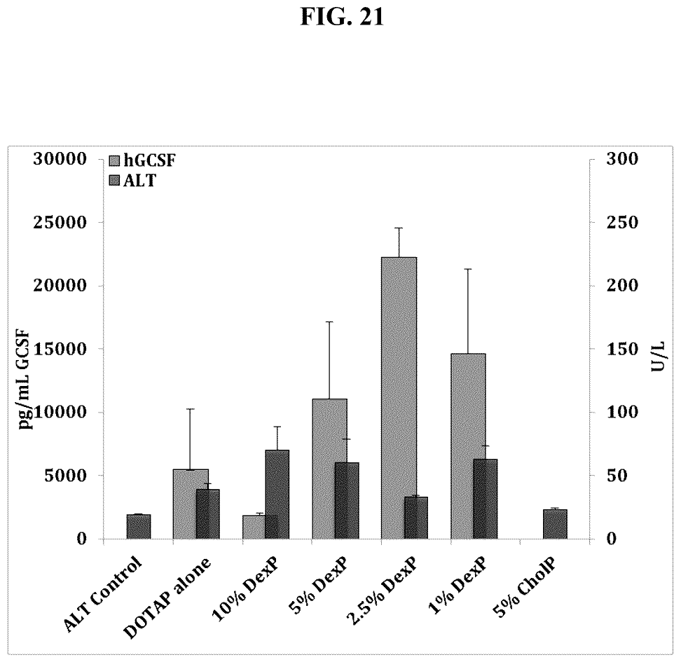

In this Example, three mice were injected per group. Each mouse received a single IV injection of 900 nmoles of either pure DOTAP SUV liposomes or each mouse received a single IV injection of 900 nmoles of either pure DOTAP SUV liposomes or liposomes containing indicated mole % s of dexamethasone covalently linked to palmitate (DexP) incorporated into the liposome bilayer. Incorporation of 5% cholesteryl palmitate (CholP) into the liposome bilayer served as a control. This was followed two minutes later by a single IV injection of 90 μg of plasmid DNA encoding hG-CSF. Serum levels of hG-CSF were determined by ELISA 24 hours following injection and ALT measurements were assayed at the UC Davis Comparative Pathology lab. As shown in FIG. 21, at 24 hours following sequential injection, toxicity as measured by ALT levels is 2-3 fold higher than seen in animals that were mock injected with lactated ringer's solution only (ALT Control). Incorporation of 2.5% dexamethasone palmitate (DexP) into the liposome bilayer produced a dual effect of increasing peak expression of hG-CSF as well as reducing ALT levels to within 1.5 fold of background (normal) levels at 24 hours.

### Example 7

### DexP Reduces Toxicity and Increases Expression

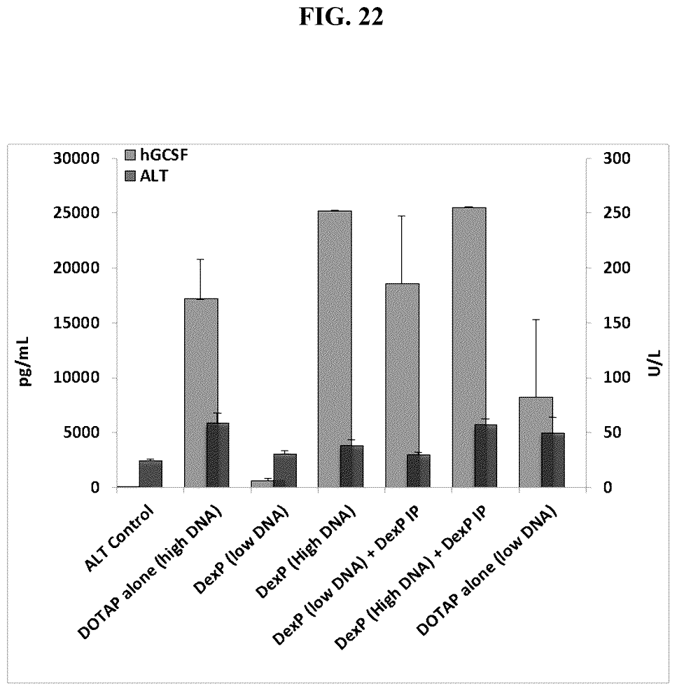

In this Example, three mice were injected per group. Each mouse received a single IV injection of 900 nmoles of either pure DOTAP SUV liposomes or liposomes containing 2.5 mole % of dexamethasone covalently linked to palmitate (DexP) incorporated into the liposome bilayer of DOTAP liposomes. This was followed two minutes later by a single IV injection of either 130 μg (high) or 40 μg (low) of an EF-1 driven plasmid DNA vector encoding hG-CSF. Two groups were treated two hours prior to IV injection with an IP injection of 1 umole of dexamethasone palmitate. Serum levels of hG-CSF were determined by ELISA 24 hours following injection and ALT measurements were assayed at 24 hours by the UC Davis Comparative Pathology lab. As shown in FIG. 22, sequential injection of 130 μg of DNA produced significantly higher hG-CSF protein levels than 40 μg of DNA 24 hours later. Inclusion of 2.5 mole % dexamethasone palmitate in the liposomes at either DNA dose further increased hG-CSF protein levels. In addition, incorporation of dexamethasone palmitate in liposomes reduced ALT levels to within 1.5 fold of background (normal) levels, even at the much higher DNA dose.

### Example 8

### Pre and Post Dex Injection

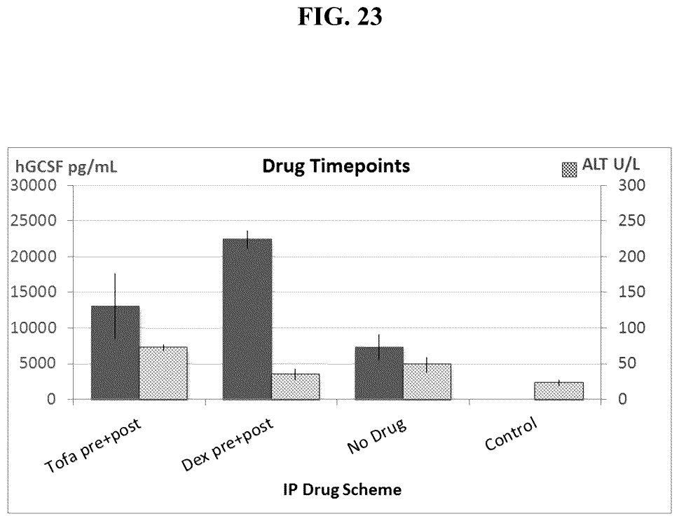

In this Example, mice were pre-injected IP with 15 mg/kg of Tofacitinib or 40 mg/kg of Dexamethasone, followed 2 hours later by sequential IV injections of 900 nmol DOTAP SUV, then 70 μg of a CPG-free, EF-1 driven hG-CSF plasmid vector. Another IP injection of Tofacitinib or Dexamethasone was administered 2 hours after injection of DNA. As shown in FIG. 23, administration of dexamethasone prior to, as well as following, sequential cationic liposome then DNA injection both significantly increased HuG-CSF protein levels while concurrently reducing toxicity within 1.5 fold of background (normal) levels. In contrast, pre- and post-injection of the immunosuppressive agent Tofacitinib did neither.

### Example 9

### Lipid to DNA Ratio

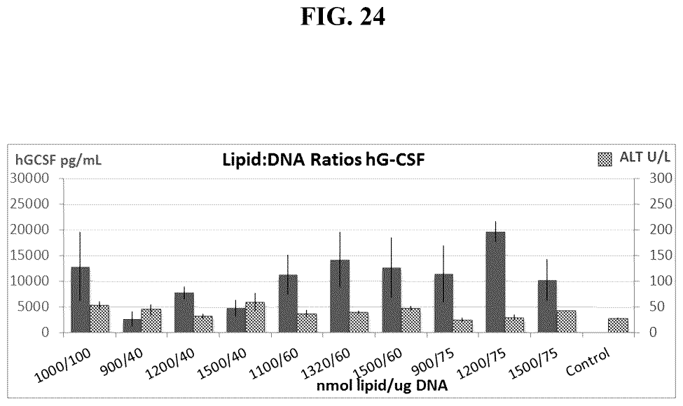

In this Example, three mice per group were given IV injections of 900, 1000, 1200, or 1500 nmols of DOTAP SUV liposomes containing 2.5 mole % of dexamethasone covalently linked to palmitate (DexP) incorporated into the liposome bilayer of DOTAP liposomes, suspended in Lactated Ringer's (LR) to a final volume of 100 uL per injection, followed 2 minutes later by 40, 60, or 75 μg of a CPG-free, EF-1 driven, hG-CSF plasmid vector at 100 uL per injection. Mock-injected mice received LR only without lipid or DNA. Serum levels of hG-CSF protein and ALT were assayed 24 hours later. As shown in FIG. 24, hG-CSF protein and ALT levels of mice sequentially injected with DOTAP SUV lipid to plasmid DNA (nmole lipid: mg DNA) ratios lower than 26:1 produced significantly higher hG-CSF protein levels while preventing toxicity, as documented by producing ALT levels either within 1.5 fold of or equal to background (normal) levels in control mice that received neither lipid nor DNA injection.

### Example 10

### Rituximab Expression

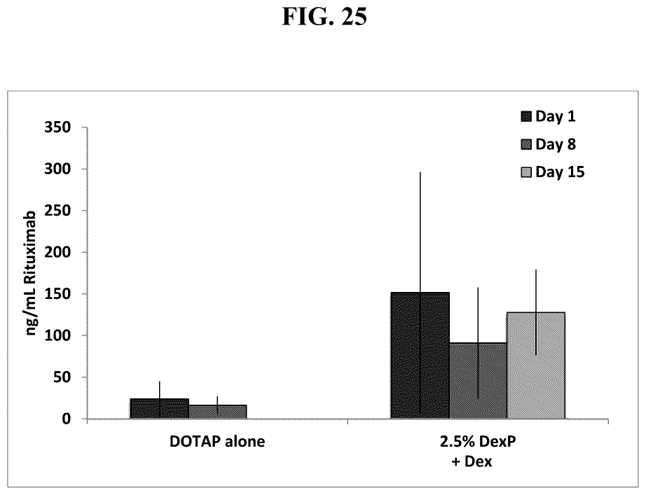

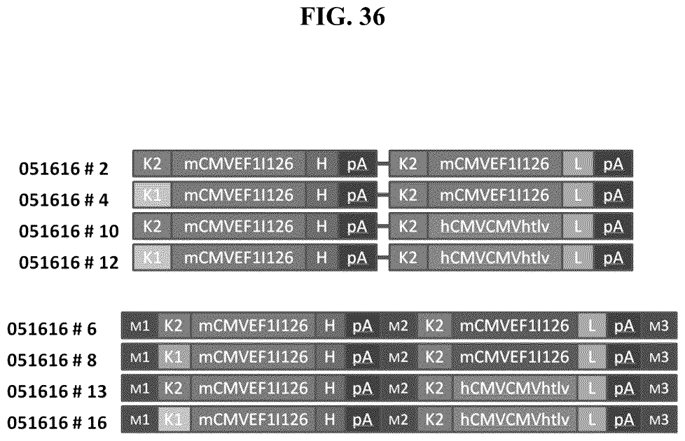

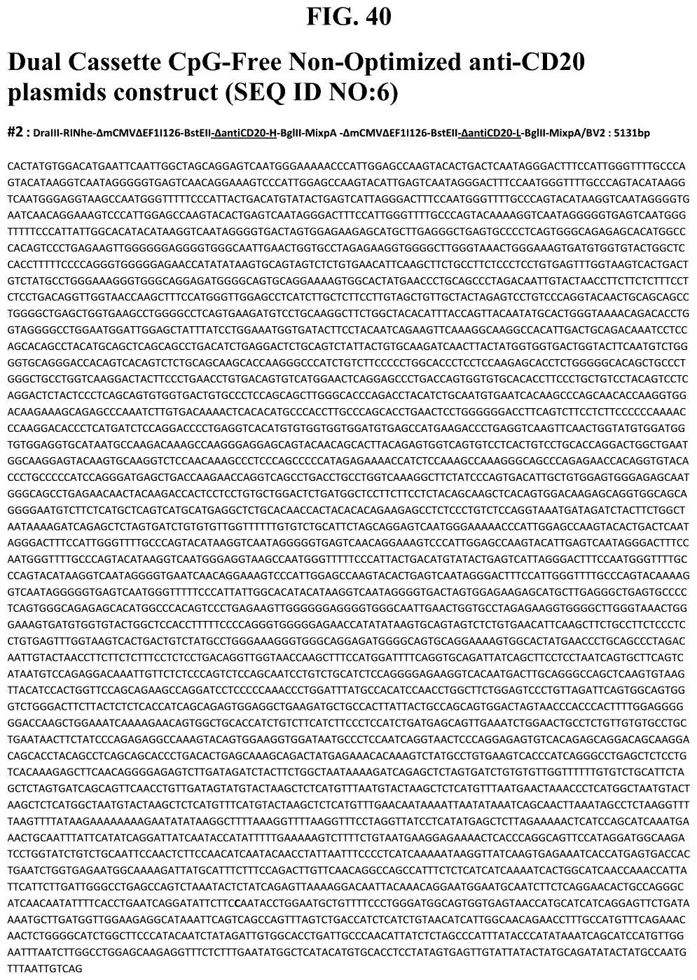

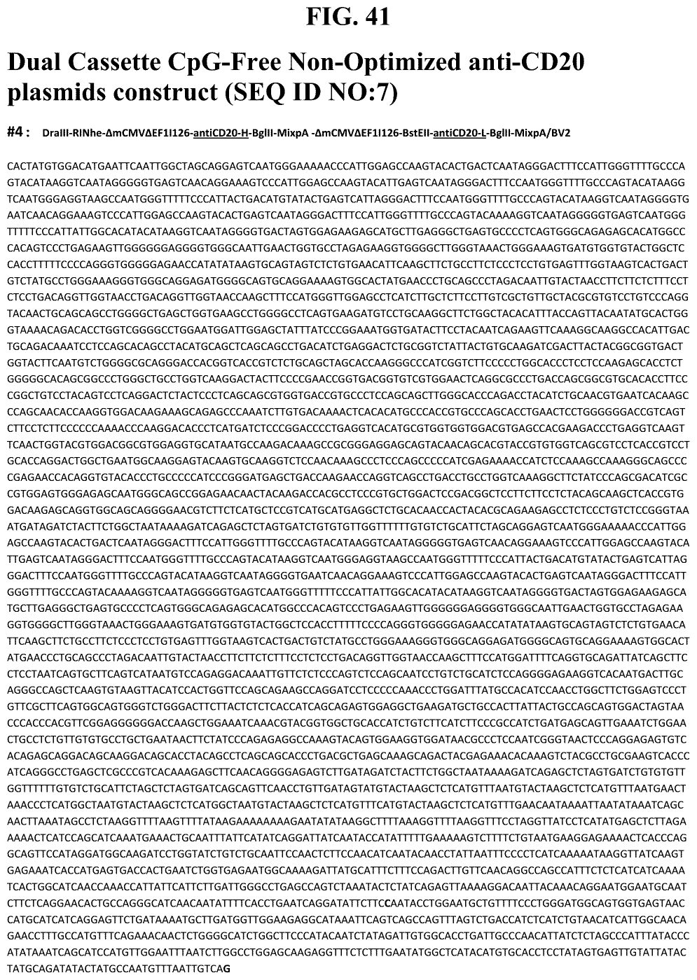

In this Example, three mice were injected per group. Each mouse received a single IV injection of 1000 nmoles of either pure DOTAP cationic liposomes or liposomes containing 2.5 mole % of dexamethasone covalently linked to palmitate (DexP) incorporated into the liposome bilayer of DOTAP liposomes. This was followed two minutes later by a single IV injection of 100 μg of a dual cassette, single plasmid DNA vector encoding the Rituximab heavy and light chains (see constructs in FIGS. 36, 40, and 41). Serum Rituximab levels were determined by ELISA 24 hours following injection and then at 7-day intervals. Mice were bled and serum isolated as for G-CSF. Rituximab levels were measured using an Immunoguide ELISA obtained from Eagle Biosciences, and performed according to instructions. As shown in FIG. 25, IP dexamethasone pretreatment plus incorporation of 2.5 mole % dexamethasone palmitate in DOTAP liposomes increases serum Rituximab levels by more than five fold for at least three weeks after injection.

### Example 11

### Rituximab Expression

In this Example, three mice were injected per group. Each mouse received a single IV injection of 1000 nmoles of either pure DOTAP cationic liposomes or liposomes containing 2.5 mole % of dexamethasone covalently linked to palmitate (DexP) incorporated into the liposome bilayer of DOTAP liposomes. This was followed two minutes later by a single IV injection of 100 μg of an EF-1-driven, dual cassette, single plasmid DNA vector encoding Rituximab (see constructs in FIGS. 36, 40, and 41). One group was treated two hours prior to IV injection with an IP injection of 40 mg/kg dexamethasone (Dex) and 1000 nmoles the neutral lipid (NL), DMPC. Serum Rituximab levels injected mice were determined by ELISA 24 hours following injection and at 7-day intervals thereafter. As shown in FIG. 26, all mice produced significant levels of serum Rituximab protein for at least 12 weeks following one injection. Mice receiving the combination of Dex, DexP and NL produced significantly higher serum Rituximab levels over time. These data show that a single sequential injection of a dual cassette Rituximab plasmid DNA vector can produce significant levels of serum Rituximab protein in animals for greater than 90 days.

### Example 12

### Dual Cassette, Single Plasmid Rituximab Expression

In this Example, Raji cells (1 million/sample) were incubated with mouse serum samples or recombinant Rituximab (50 ng/ml) for 1 hr at 4 C, in FACS binding buffer containing EDTA and 0.5% BSA. Following washes, samples were incubated with fluorescently labeled secondary antibody (anti-human IgG-PE) for 30 min, washed and analyzed using an Accuri flow cytometer. Between 3500-5000 events were recorded for each sample. The experiment was repeated twice with similar results.

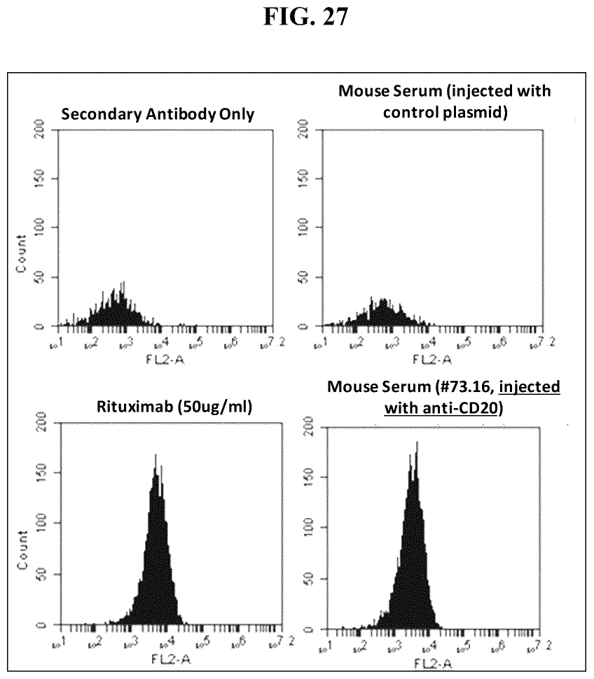

FIG. 27 shows a FACS plots display fluorescence intensity for four experimental conditions. The upper panels show samples containing mouse serum from control (HuG-CSF) DNA plasmid vector injected mice or secondary antibody alone, which display low, background levels of fluorescence in the PE channel (˜300). The lower two panels show recombinant Rituximab protein (left panel) and mouse serum following Rituximab plasmid DNA vector administration (right panel). Both samples show fluorescence intensities over 10 fold higher than the background as shown in Table 4 below, demonstrating that Rituximab present in the mouse serum binds to target CD20-expressing human Raji B cells to an extent similar to recombinant Rituximab protein. Thus, the Rituximab present in mouse serum six weeks after injection of a dual cassette, single plasmid Rituximab DNA vector binds CD20+target human B cells in a fully functional manner.

TABLE 4

| | Mean Fluorescence |
|---|---|
| Sample designation | Intensity |
| Secondary antibody | 331.86 |
| alone | |
| Mouse Serum (injected | 279.32 |
| with control plasmid) | |
| Recombinant Rituximab | 5781.87 |
| (50 ug/ml) | |
| Mouse Serum (injected | 3532.40 |
| with anti-CD20 plasmid) | |

### Example 13

### Functional Rituximab is Expressed

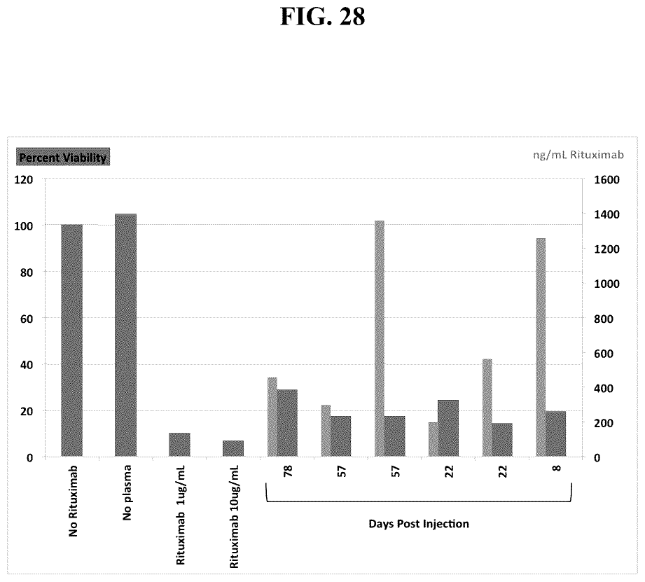

In this Example, Raji cells (5×10⁴ cells/well) were plated in 96 well plates using RPMI+10% FBS medium. Next day cells were incubated with Rituximab (1, 10 μg/ml) or mouse serum samples (20 μl/well) for 1 hour at room temperature. Twenty ul of pooled normal human plasma (Innovative Research) was then added to all wells (except the Rituximab control condition) and the plates incubated for another 12 h at 37 C. Cell viability was measured using the Promega Cell titer glo reagent according to the manufacturer's instructions. In FIG. 28, values are shown as percentage change from the control conditions in which serum from mice injected with a huG-CSF DNA plasmid DNA vector was used. Individual mouse sera were tested from five different mouse groups that received a single sequential injection of a dual cassette, single plasmid Rituximab DNA vector from 8 to 78 days prior to serum collection.

Results of this Example are shown in FIG. 28. Sera from mice previously injected with a dual cassette, single plasmid Rituximab DNA vector were analyzed first by ELISA to quantitate serum Rituximab concentrations. Adding these Rituximab-containing sera in a cell lysis assay then showed that they lyse CD-20+ human Raji B cells in a manner comparable to recombinant Rituximab (Invivogen). Moreover, functional serum Rituximab protein with documented lytic activity was isolated from animals across five separate injection experiments over a eleven-week period, demonstrating its reproducible lytic efficacy over time.

### Example 14

### Enhanced Expression of Rituximab

In this Example, two, 250 gm Sprague-Dawley female rats per group were were first pre-injected with 40 mg/kg dexamethasone, then sequentially injected with 4400 nmol of DOTAP SUV liposomes containing 2.5 mole % of dexamethasone covalently linked to palmitate (DexP) incorporated into the liposome bilayer, with or without 4400 nmol of neutral DMPC lipid, then 360 μg of a dual cassette, single plasmid DNA vector containing an EF1-driven Rituximab cDNA (see constructs in FIGS. 36, 40, and 41). Serum levels of human Rituximab protein were assessed at 7-day intervals following injection. FIG. 29 shows significantly higher levels of serum Rituximab protein were produced in rats also receiving neutral lipid for at least 15 days following a single IV injection.

### Example 15

### Codon-Optimized Rituximab Expression

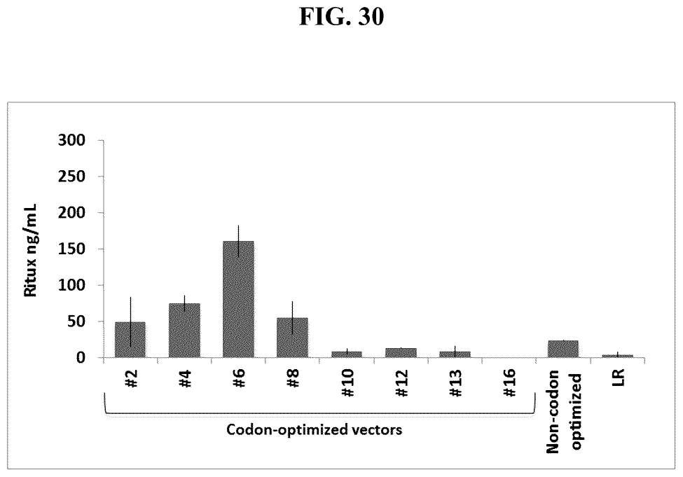

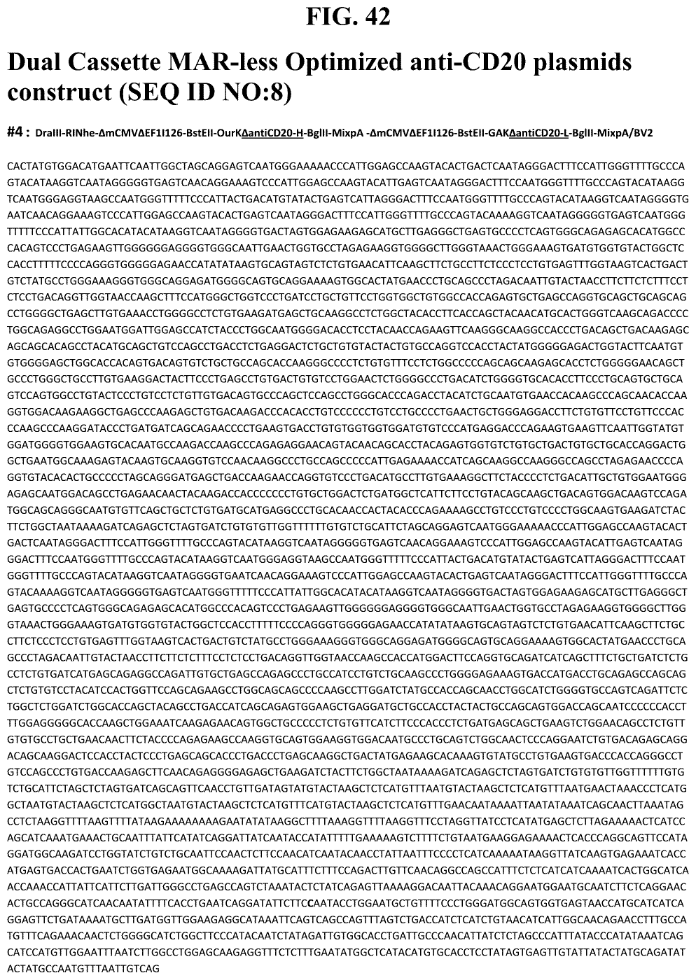

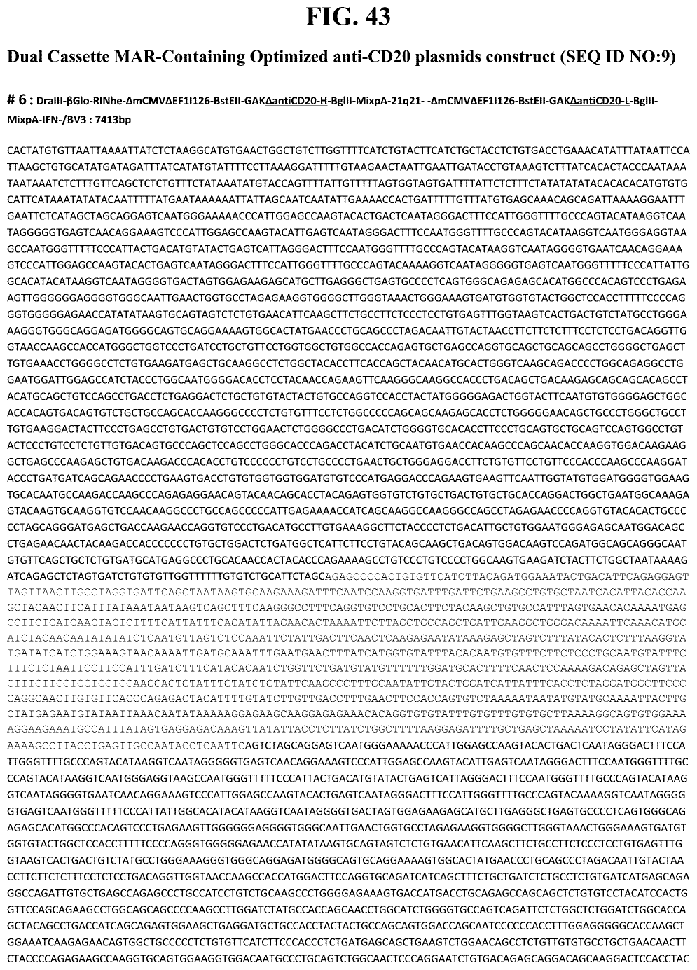

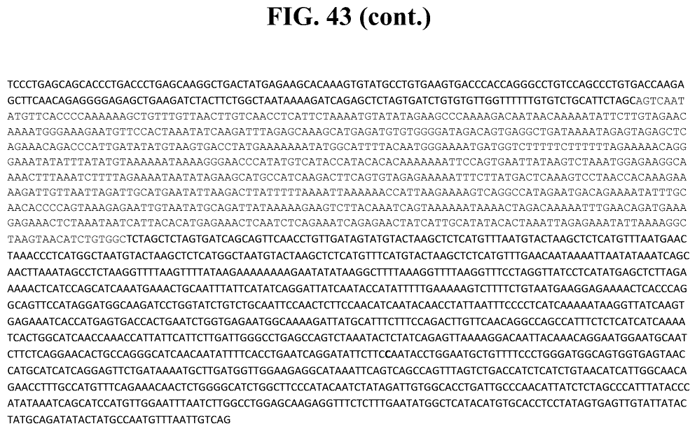

In this Example, three mice were injected per group. Each mouse received a single IV injection of 1000 nmoles of liposomes containing 2.5 mole % of dexamethasone covalently linked to palmitate (DexP) incorporated into the liposome bilayer of DOTAP liposomes. This was followed two minutes later by a single IV injection of 100 μg of dual cassette, single plasmid EF-1-driven DNA vector encoding Rituximab (see constructs in FIGS. 36, 42, and 43). Numbered plasmids (in FIG. 30) were codon-optimized versions of the original, CpG-free but not codon optimized Rituximab DNA sequence. Serum levels of Rituximab were determined by ELISA 24 hours following injection. FIG. 30 shows that at 24 hours following sequential injection, codon-optimized DMA vector 6 produced significantly higher levels of serum Rituximab protein than non-codon optimized rituximab DNA vectors.

### Example 16

### Codon-Optimized Rituximab Expression

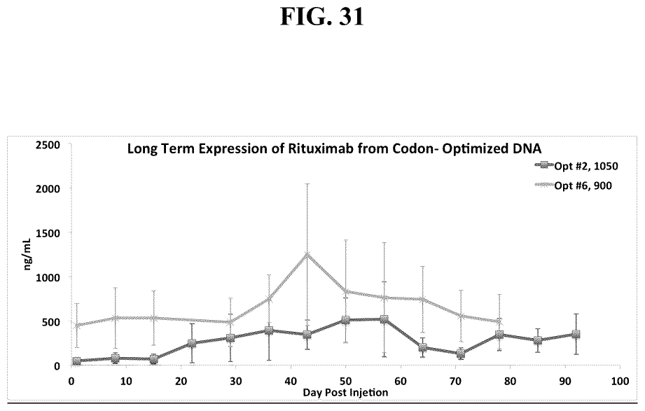

In this Example, three mice were injected per group. Each mouse received a single IV injection of DOTAP cationic liposomes (900 nmoles or 1050 nmoles as indicated) containing 2.5 mole % of dexamethasone covalently linked to palmitate (DexP) incorporated into the liposome bilayer. This was followed two minutes later by a single IV injection of 75 μg of a dual cassette, codon-optimized single plasmid DNA encoding Rituximab (see constructs in FIGS. 36, 42, and 43). Both groups were treated two hours prior to IV injection with an IP injection of 40 mg/kg dexamethasone. Serum levels of Rituximab were determined by ELISA 24 hours following injection and at 7-day intervals thereafter. FIG. 31 shows that one sequential IV injection of codon-optimized dual cassette, single plasmid Rituximab DNA vectors produces extended serum Rituximab levels for at least the next 60 days. Serum Rituximab levels rise over time after a single IV sequential injection.

### Example 17

### Valproic Acid and Theophylline Increase Protein Expression

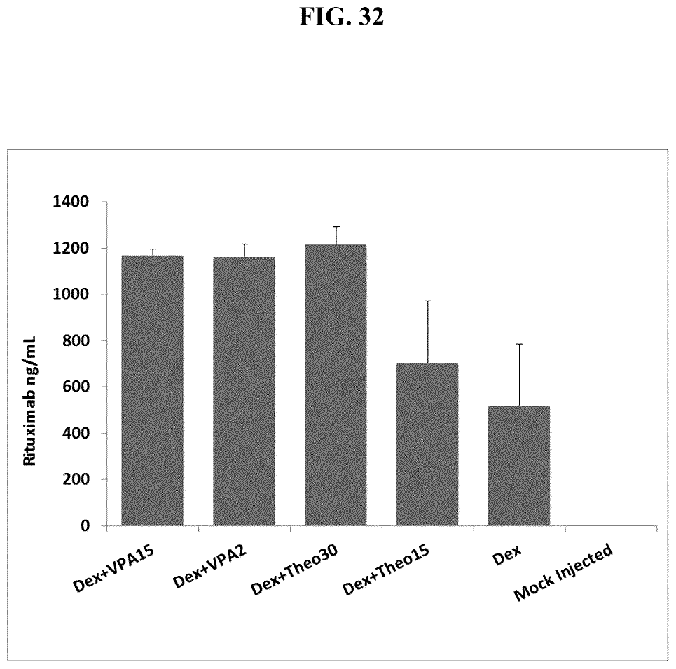

In this Example, three mice were injected per group. Each mouse received a single IV injection of 1050 nmoles of liposomes containing 2.5 mole % of dexamethasone covalently linked to palmitate (DexP) incorporated into the liposome bilayer of DOTAP liposomes. This was followed two minutes later by a single IV injection of 75 μg of dual cassette plasmid DNA encoding Rituximab. All groups were treated two hours prior to IV injection with an IP injection of 40 mg/kg dexamethasone. Where indicated in FIG. 32, animals were also pretreated by IP injection of 15 mg/kg Valproic Acid (VPA), 2 mg/kg VPA, 30 mg/kg Theophylline (Theo) or 15 mg/kg Theo. Serum Rituximab levels were determined by ELISA 24 hours following injection. FIG. 32 shows serum Rituximab levels produced were significantly increased by pre-treatment with the drugs Valproic Acid or Theophylline, thus providing a framework for further enhancing protein levels without altering the dose of lipid or DNA.

### Example 18

### Dual or Single Cassette Rituximab Expression

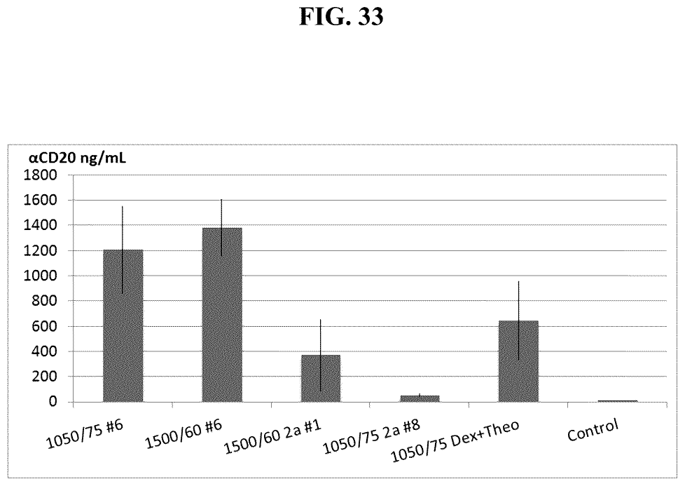

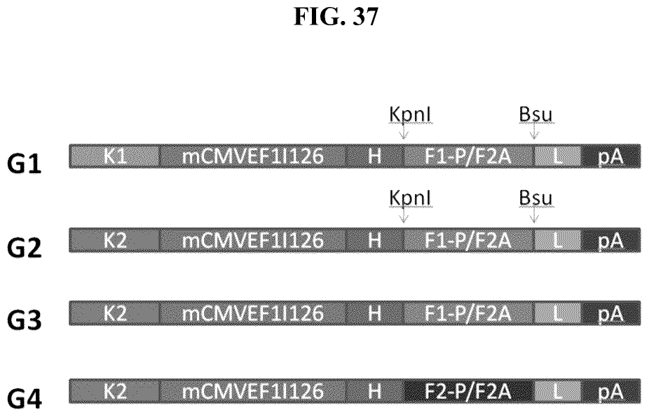

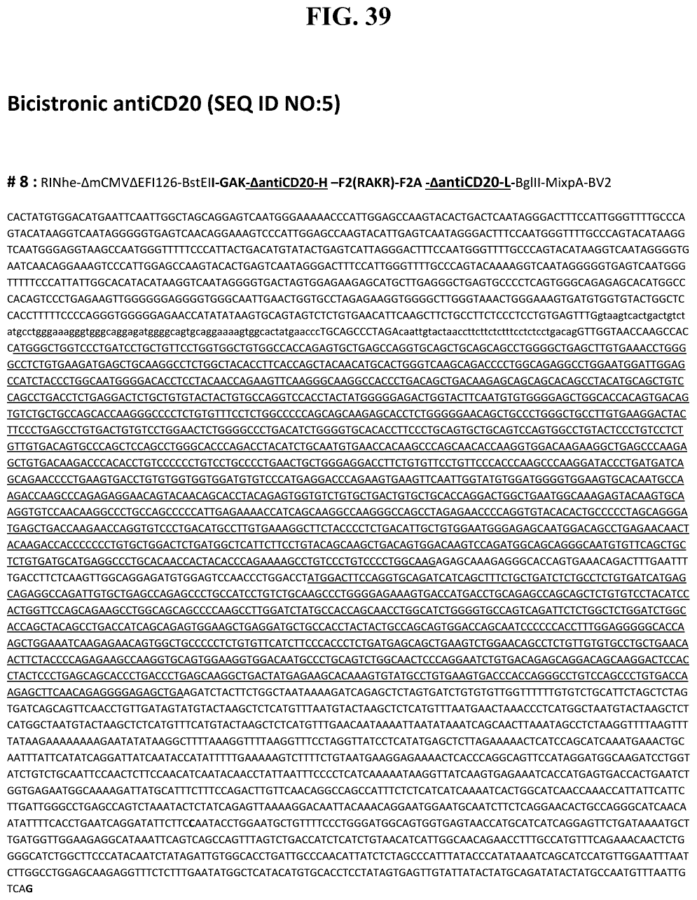

In this Example, mice were pre-injected with 40 mg/kg Dexamethasone IP. Two hours later, they were sequentially injected with either 1050 or 1500 nmols of DOTAP SUV liposomes containing 2.5 mole % of dexamethasone covalently linked to palmitate (DexP) incorporated into the liposome bilayer, followed by 60 μg or 75 μg of plasmid DNA. Plasmid DNA constructs injected were either codon optimized, double-cassette, single plasmid DNA vectors (see constructs in FIGS. 36, 42, and 43) or codon optimized single-cassette plasmids (see constructs in FIGS. 36, 37, and 39) containing Rituximab heavy and light chain sequences separated by a 2A self-cleaving peptide DNA sequence. Serum Rituximab levels were determined by ELISA 24 hours following injection. FIG. 33 shows that at 24 hours following sequential injection, mice that received 2A peptide containing single cassette vectors encoding Rituximab produced serum levels approaching 400 ng/ml, approximately one-third the level produced by the dual cassette vector. Thus, significant Rituximab serum levels can be produced by either dual- or single-cassette, 2A peptide-containing DNA vectors.

### Example 19

### Lipid to DNA Ratio and Rituximab Expression

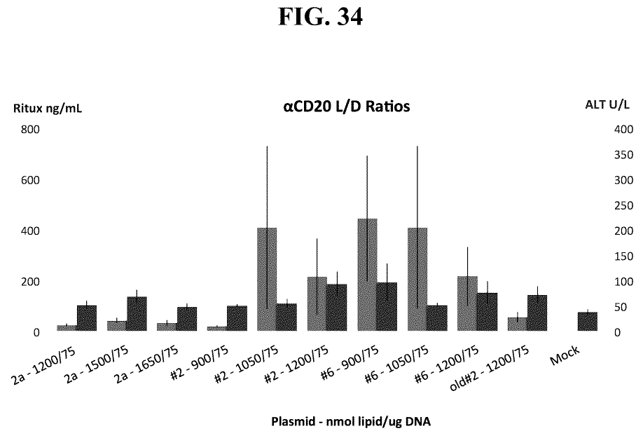

In this Example, three mice per group were given IV injections of 900, 1050, 1200, 1500 or 1650 nmols of DOTAP SUV liposomes containing 2.5 mole % of dexamethasone covalently linked to palmitate (DexP) incorporated into the liposome bilayer, suspended in Lactated Ringer's (LR) to a final volume of 100 uL per injection, followed 2 minutes later by 75 μg of a CPG-free, dual cassette, single plasmid Rituximab vector at 100 uL per injection. Mock-injected mice received LR only without lipid or DNA. Serum levels of Rituximab protein and ALT were assayed 24 hrs later. FIG. 34 shows Rituximab protein and ALT levels of mice sequentially injected with DOTAP SUV lipid to plasmid DNA (nmole lipid: mg DNA) ratios lower than 15:1 produced significantly higher Rituximab protein levels while producing serum ALT levels within 1.5 fold of background (normal) ALT levels in control mice that received neither lipid nor DNA injection.

### Example 20

### Factor IX Expression with Valproic Acid or Theophylline

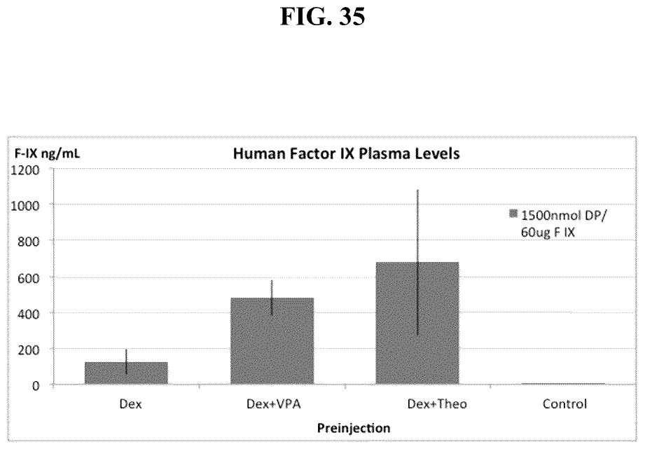

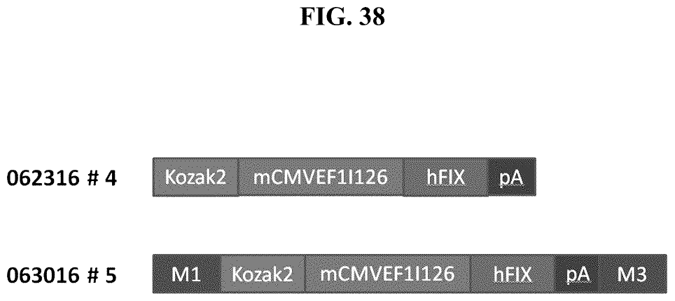

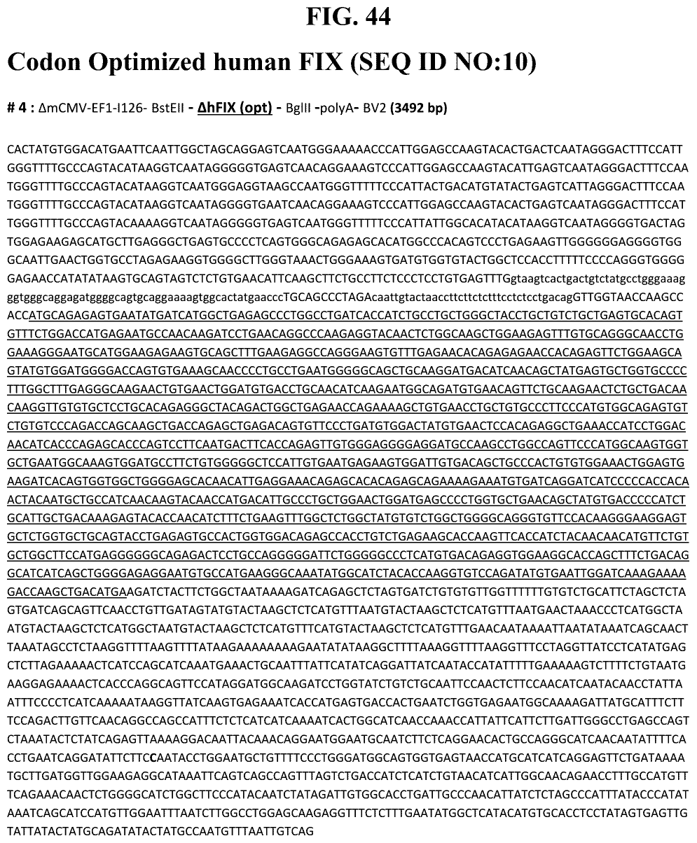

In this Example, three mice were injected per group. Each mouse received a single IV injection of 1500 nmoles of DOTAP SUV liposomes containing 2.5 mole % of dexamethasone covalently linked to palmitate (DexP) incorporated into the liposome bilayer. This was followed two minutes later by a single IV injection of 60 μg of a codon optimized, EF-1-driven single cassette DNA vector encoding the human factor IX cDNA (see constructs in FIGS. 38 and 44). All groups were treated two hours prior to IV injection with an IP injection of 40 mg/kg dexamethasone. Where indicated, animals were also pretreated by IP injection of 2 mg/kg Valproic Acid (VPA) or 30 mg/kg Theophylline (Theo). Serum human factor IX levels were determined by ELISA 24 hours following injection. Each mouse was bled as for G-CSF. Blood was collected into tubes containing Potassium EDTA or Sodium Citrate to prevent coagulation and centrifuged to obtain plasma. An AssayPro ELISA specific to human Factor IX was used according to manufacturer's instructions to measure Factor IX expression. FIG. 35 shows serum human factor IX levels produced at 24 hrs were significantly higher in mice receiving the human factor IX DNA vector plus pre-treatment with either Valproic Acid or Theophylline.

### Example 21

### Size Determination of Liposomes

In this Example, the sizes of various liposomes were determined. In particular, the liposomes in Table 5 were prepared in 5% w/w glucose, and the size was determined using quasi elastic laser light scattering. The Z-Average particle size of these DOTAP liposomes is shown in Table 5.

TABLE 5

| Liposome Type | Z-Average Particle Size (nm) |
|---|---|
| DOTAP Multilamellar Liposomes (MLV) | 339 |
| DOTAP 0.1 micron Extruded MLV | 146 |
| DOTAP Sonicated Liposomes (SUV) | 74 |

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A system comprising:
a) a first composition comprising a first amount of polycationic structures liposomes, wherein said first composition is free, or essentially free, of nucleic acid molecules; and
b) a second composition comprises non-viral expression vectors,
wherein said non-viral expression vectors are CpG-free or CpG-reduced,
wherein each of said non-viral expression vectors comprises a first nucleic acid sequence encoding: i) a first therapeutic protein, and/or ii) a first biologically active nucleic acid molecule; and
at least one of the following:
i) wherein the ratio of said first amount of said polycationic structures to said non-viral expression vectors is 5:1 to 25:1;
ii) wherein 2.0% to 6.0% of said first composition comprises dexamethasone or dexamethasone palmitate;
iii) wherein said first composition further comprises neutral lipid; and
iv) wherein said polycationic structures comprise empty liposomes, and wherein said empty liposomes present in said first composition have a z-average diameter of about 20-85 nm.

2. The system of claim 1, wherein said wherein said polycationic structures comprise empty liposomes, and wherein said empty liposomes present in said first composition have a z-average diameter of about 20-85 nm.

3. The system of claim 1, wherein said non-viral expression vectors each further comprise an regulating nucleic acid sequence, wherein said regulating nucleic acid sequence reduces the duration of expression of said first nucleic acid sequence that would occur in the absence of said regulating nucleic acid sequence.

4. The system of claim 3, wherein said regulating nucleic acid sequence is selected from the group consisting of: a promoter, an enhancer, a second nucleic acid sequence encoding a second protein, and/or a second biologically active nucleic acid molecule,

5. The system of claim 1, wherein said first amount of polycationic structures in said first composition comprises a mixture of cationic lipid and neutral lipid that reduces the expression of said first therapeutic protein and/or first biologically active nucleic acid molecule compared to such expression when only said cationic lipid is employed in said method.

6. The system of claim 1, wherein said first nucleic acid sequence encodes said therapeutic protein, and wherein said therapeutic protein is selected from the group consisting of: human G-CSF, Rituximab, and human Factor IX.

7. The system of claim 1, wherein said non-viral expression vectors comprise a first nucleic acid sequence encoding said first therapeutic protein.

8. The system of claim 1, wherein said non-viral expression vectors comprise a first nucleic acid sequence encoding said first therapeutic protein, wherein said first therapeutic protein comprises a monoclonal antibody light chain.

9. The system of claim 1, wherein said non-viral expression vectors comprise a first nucleic acid sequence encoding said first therapeutic protein, wherein said first therapeutic protein comprises a monoclonal antibody heavy chain.

10. The system of claim 1, wherein said non-viral expression vectors comprise: i) a first nucleic acid sequence encoding said first therapeutic protein, wherein said first therapeutic protein comprises a monoclonal antibody heavy chain, and ii) a second nucleic acid sequence encoding a second therapeutic protein, wherein said second therapeutic protein comprises a monoclonal antibody light chain.

11. The system of claim 1, wherein said non-viral expression vectors comprise a first nucleic acid sequence encoding said first biologically active nucleic acid molecule.

12. The system of claim 1, wherein said non-viral expression vectors comprise a first nucleic acid sequence encoding said first biologically active nucleic acid molecule, wherein said first biologically active nucleic acid molecule comprises a shRNA sequence.

13. The system of claim 1, wherein said non-viral expression vectors comprise a first nucleic acid sequence encoding said first biologically active nucleic acid molecule, wherein said first biologically active nucleic acid molecule comprises a miRNA sequence.

14. The system of claim 1, wherein said non-viral expression vectors comprise a first nucleic acid sequence encoding said first biologically active nucleic acid molecule, wherein said first biologically active nucleic acid molecule comprises an antisense sequence or ribozyme.

15. The system of claim 1, wherein said non-viral expression vectors comprise a first nucleic acid sequence encoding said first biologically active nucleic acid molecule, wherein said first biologically active nucleic acid molecule comprises a CRISPR single guide RNA sequence.

16. The system of claim 1, wherein said at least one of the following is i) wherein the ratio of said first amount of said polycationic structures to said non-viral expression vectors is 5:1 to 25:1.

17. The system of claim 1, wherein said at least one of the following is ii) wherein 2.0% to 6.0% of said first composition comprises dexamethasone or dexamethasone palmitate.

18. The system of claim 1, wherein said at least one of the following is iii) wherein said first composition further comprises neutral lipid.

19. The system of claim 1, wherein said at least one of the following is iv) wherein said polycationic structures comprise empty liposomes, and wherein said empty liposomes present in said first composition have a z-average diameter of about 20-85 nm.
